(19) United States
(12) Patent Application Publication
(10) Pub. No.: US 2015/0183835 A1
(43) Pub. Date: Jul. 2, 2015

(54) STABILIZED GP120

(71) Applicant: Novartis AG, Basel (CH)

(72) Inventors: Andrea Carfi, Cambridge, MA (US); Antu Dey, Holly Springs, NC (US); Aemro Kassa, Athens, GA (US); Indresh K. Srivastava, Meriden, CT (US)

(21) Appl. No.: 14/408,466

(22) PCT Filed: Jun. 17, 2013
(86) PCT No.: PCT/EP2013/062553

§ 371 Date: Dec. 16, 2014

Related U.S. Application Data
(60) Provisional application No. 61/661,050, filed on Jun. 18, 2012.

(51) Int. Cl. C07K 14/005; C12N 7/00; A61K 39/21

(52) U.S. Cl. CPC C07K 14/005; A61K 39/21; C12N 7/00; C12N 2740/16122; C12N 2740/16134; C07K 2319/00; C12N 2760/16022; C12N 2740/16171

(57) ABSTRACT

The present invention provides an isolated polypeptide comprising an HIV gp120 polypeptide or soluble gp140 polypeptide stabilized in a conformation which exposes both CD4-bound and CD4-binding site epitopes. The invention also provides immunogenic compositions and methods of treating and preventing infection with HIV.

This patent application claims priority from U.S. provisional patent application 61/661,050 filed Jun. 18th 2012, the complete contents of which are incorporated herein by reference.

### TECHNICAL FIELD

The present invention is in the field of HIV, and in particular in the field of HIV vaccines.

### BACKGROUND ART

The HIV/AIDS pandemic has spread all over the world and by the end of 2007 more than 60 million people were infected with HIV-1 [1]. Despite the large amount of resources which have been targeted at fighting the pandemic, there is still no vaccine available against HIV-1. Although the human body generates a robust immune response against initial HIV-1 infection, many of these antibodies fail to neutralize the virus [2]. One reason is the presence of multiple levels of defence shielding the virus against neutralizing antibodies. These include high degree of sequence variability, carbohydrate masking, occlusion of epitopes by multimer formation and conformational masking [3].

Despite a large degree of variability, the virus needs to maintain conserved sites that can recognize and bind cellular receptors. These conserved receptor binding sites in HIV-1 gp120, which include CD4-binding site and CD4-induced (co-receptor) binding sites, which include the CD4-binding site and the CD4-bound epitopes respectively, are vulnerable targets for antibody mediated neutralization and are thus an attractive target for vaccine production. However, these conserved sites, and the epitopes at these sites, are conformationally masked and are therefore not accessible to neutralizing antibodies.

Attempts have been made in the past to stabilize gp120 in a conformation that exposes the conserved regions of the protein by stabilizing the inner and outer domain contacts. For example, previous structure-guided attempts to stabilize gp120 in receptor bound conformation utilized various approaches including disulfide bridges and cavity filling mutations [4], as well as the removal of immunodominant regions such as the V1/V2 and V3 loops prior to stabilization[5]. The largest degree of stabilization was achieved by the formation of disulfide bridges after targeted substitution with cysteine residues [6 and 7]. However, while these disulfide bonds stabilized the structure of gp120 in a conformation that exposed the CD4-bound epitopes in gp120, it failed to expose the CD4-binding site epitopes, as measured by affinity of CD4-binding site antibodies. In addition to this, strategies that involve the removal of the variable loops are also disadvantageous, as the variable loops are involved in regulation of the quaternary structure of gp120 [8].

### DISCLOSURE OF THE INVENTION

The structure of the inner domain of HIV-1 gp120 is missing from published gp120 structures [4, 9 and 10]. However, the interactions of the layers of the inner domain are important in the conformational changes that take place in gp120 upon CD4 ligand binding. The present invention relates to the stabilization of inter layer interactions in the inner domain of HIV-1 gp120 and soluble HIV-1 gp140.

### HIV Structure

The newly elucidated structure of the inner domain of gp120 shows that in CD4-bound state, the inner domain is organized into 3 layers, consisting of layers 1, 2 and 3, projecting from a β-sandwich structure towards the target cell membrane (FIG. 1) [11-13]. The inner domain is made up of amino acids corresponding to amino acids 1-248 and 458-497 of HIV gp120 from SF162 strain (SEQ ID NO: 2). Layer 1 is made up of amino acids corresponding to amino acids 42-76 of HIV gp120 from SF162 strain. Layer 2 is made up of amino acids corresponding to amino acids 90-117 and 192-223 of HIV gp120 from SF162 strain. Layer 3 is made up of amino acids corresponding to amino acids 239-249 and 459-470 of HIV gp120 from SF162 strain. The inner domain has been identified as a region of gp120 that undergoes extensive conformational changes as the polypeptide transitions from the un-liganded to CD4-bound state[14, 15]. The conformational changes in the inner domain that take place upon CD4 binding contribute to two downstream events required for successful fusion of the HIV virus to a target cell. These events are:
1. Stabilization of the initial weak CD4-contacts, and
2. Exposure of a conserved site on gp120 for co-receptor binding, i.e. exposure of the CD4-bound epitopes.

In the CD4-bound state, the three layers of the inner domain of gp120 come together through specific interlayer interactions and stabilize the CD4-bound conformation of gp120.

Interactions between layers 1 and 2 are mediated through a number of residues which have been identified by site-directed mutagenesis [11, 12]. These interactions between layers 1 and 2 form a unique structure, similar to a “collar”, around the inner domain and stabilize the region[8]. Disruption of these inter-layer interactions leads to destabilization of the CD4-bound conformation of gp120 resulting in reduced affinity to CD4 [11]. Similarly, mutations that reduced interaction between the layers of the inner domain also prevent binding of gp120 to small molecule CD4-mimetics, such as NBD-556 which require CD4-bound conformation of the gp120 for a high affinity interaction[11, 16]. Binding of antibodies, such as 17b, 48d and 412d [17, 18 and 27] that recognize the CD4-induced conserved binding sites on gp120 were also dramatically reduced by mutations that prevented inter-layer interaction in the inner domain [11]. In general, reduced inter-layer interactions lead to reduced exposure of the conserved binding sites (i.e. CD4 and co-receptor binding sites) and therefore reduce binding of ligands recognizing these sites.

In contrast to the approaches disclosed above, the present inventors have found that stabilization of the inter-layer contacts in the inner domain induces gp120 to take up a conformation in which both the CD4-bound epitopes (also referred to as CD4-induced or CD4i epitopes) and CD4-binding site epitopes are exposed. The present invention therefore provides:
An isolated polypeptide comprising an HIV gp120 polypeptide or soluble gp140 polypeptide stabilized in a conformation which simultaneously exposes both CD4-bound and CD4-binding site epitopes
An immunogenic fragment of the stabilized HIV gp120 polypeptide or soluble gp140 polypeptide which comprises both CD4-bound and CD4-binding site epitopes of HIV gp120
A trimeric polypeptide comprising three subunits, each subunit independently selected from the group consisting of an isolated polypeptide comprising an HIV gp120 polypeptide stabilized in a conformation which simultaneously exposes both CD4-bound and CD4-binding site epitopes, soluble gp140 polypeptide stabilized in a conformation which simultaneously exposes both CD4-bound and CD4-binding site epitopes, an immunogenic fragment of the stabilized HIV gp120 polypeptide which comprises both CD4-bound and CD4-binding site epitopes of HIV gp120 and soluble gp140 polypeptide which comprises both CD4-bound and CD4-binding site epitopes of HIV gp120
Polynucleotides encoding the polypeptides listed above
An immunogenic composition comprising a polypeptide, immunogenic fragment, fusion protein and/or polynucleotide of the invention
A method of generating an immune response in a subject
A method of treating or preventing HIV infection

### Stabilized HIV Gp120 and Soluble Gp140 Polypeptides

The invention provides an isolated polypeptide comprising an HIV gp120 or soluble gp140 polypeptide stabilized in a conformation which simultaneously exposes both CD4-bound and CD4-binding site epitopes. By “stabilized in a conformation which simultaneously exposes both CD4-bound and CD4-binding site epitopes” it is meant that the inner domain of the HIV gp120 or soluble gp140 polypeptide takes up the CD4-bound conformation even in the absence of the CD4 ligand. Thus, the conformationally masked epitopes that are exposed in wild-type gp120 or soluble gp140 only when bound to CD4 are constitutively exposed in the stabilized gp120 or soluble gp140 polypeptides of the invention. Furthermore, the stabilized gp120 or soluble gp140 polypeptides of the invention take up the CD4-bound conformation in the absence of CD4, and therefore the CD4 binding site epitopes can also be exposed at the same time as the CD4-bound epitopes.

An isolated polypeptide comprising an HIV gp120 or soluble gp140 polypeptide stabilized in a conformation which simultaneously exposes both CD4-bound and CD4-binding site epitopes according to the invention is a polypeptide which binds specifically to
(i) an anti-gp120 CD4 binding site antibody, i.e. an antibody specific for the CD4 binding site in gp120,
(ii) an antibody specific for a CD4-induced conserved binding site in gp120, and
(iii) optionally, a further anti-gp120 or anti-soluble gp140 antibody.

By “binds specifically”, it is meant that the antibodies bind to a polypeptide of the invention with substantially greater affinity than to BSA. Preferably, the affinity is at least 100-fold, 10³-fold, 10⁴-fold, 10⁵-fold, 10⁶-fold etc. greater for the polypeptides of the invention than for BSA.

Typical anti-gp120 antibodies are well known in the art and include for example B12 [19], F105 [20], 17b [17], 48d [27], 412d [21], B13 [22] and 2G12 [23], and the antibodies 46-2 (CRL-2186), 46-4 (CRL-2178), 46-5 (CRL-2184), 55-2 (CRL-2155), 55-36 (CRL-2153), 55-6 (CRL-2185) and 55-83 (CRL-2395) available from the ATCC. Anti-gp120 CD4 binding site (α-gp120-CD4BS) antibodies include monoclonal antibodies B12, F105, JL413 [24], 1795 [25], 448-D (ATCC HB-10895), 558-D (ATCC HB-10894) and 559/64-D (ATCC HB-10893) [26]. Antibodies specific for a CD4-induced conserved binding site in gp120 (α-gp120 CD4i) include 17b, 48d, 412d, and 23e (2.3E) described in reference 27. These α-gp120 CD4i antibodies can be obtained from the NIH AIDS Research & Reference Reagent Programme (https://www.aidsreagent.org/). The sequences of these antibodies have been published [28], and the nucleitide sequences are available in the IGBLAST (http://www.ncbi.nlm.nih.gov/igblast/) or ImMunoGeneTics information system (IMGT) (http://imgt.cines.fr/) databases.

In one embodiment, the gp120 polypeptide or soluble gp140 polypeptide is stabilized by one or more inter-layer contacts between the layers of the inner domain. In a particular embodiment, the stabilizing contact is made by a disulphide bond between a pair of non-naturally encoded cysteine residues.

The stabilizing contact may be made between layers 1 & 2, 2 & 3 or both layers 1 & 2 and 2 & 3.

In order for a disulfide bridge to be formed between two cysteine residues, the side chains have to be at the correct distance and in the correct orientation with respect to each other. Examination of all residues that lie at the interface of layers 1, 2 and 3 of the inner domain led to the identification of residues that can be targeted for substitution with paired cysteine residues for disulfide formation. The identification of appropriate residues was based on the crystal structure of gp120 [13]. Pairs of residues were considered suitable if they were predicted to have a Cβ-Cβ distance of between 3.9 Å-5.8 Å.

On this basis, the following pairs of residues were identified for substitution with cysteine to form stabilizing disulphide bridges between layers: V59 & S109, V95 & W465, V95 & R462, V95 & L469, and/or H99 & R462 (residue numbering according to the SF162 strain). Amino acid position in gp120 from other HIV-1 strains that are equivalent to the amino acid pairs listed above in the gp120 polypeptide from SF162 strains can be identified, for example, by aligning the gp120 polypeptide sequence from a second strain with the SF162 gp120 polypeptide sequence. FIG. 5 shows an alignment of gp120 from the SF162 and Hxb2 strains of HIV-1. The corresponding residues in Hxb2 gp120 are identified in grey numbering. The corresponding pairs of residues for substitution with cysteine to form stabilizing disulphide bridges are V65 & S115, V101 & W479, V101 & R476, V101 & L483, and H105 & R476 in Hxb2.

Similarly, the corresponding residues in soluble gp140 polypeptides can be identified by aligning the gp120 polypeptide sequence with gp140 sequences. The SF162 soluble gp140 amino acid sequence is given in SEQ ID NO: 23.

Thus, the invention provides an isolated polypeptide comprising an HIV gp120 polypeptide or soluble gp140 polypeptide stabilized in a conformation which simultaneously exposes both CD4-bound and CD4-binding site epitopes, wherein the stabilization is achieved by inter-layer disulphide bonds in the inner domain and the disulphide bonds are formed by cysteine residues at positions equivalent to one or more of the following pairs in the wild-type SF162 HIV gp120 polypeptide: V59 & S109, V95 & W465, V95 & R462, V95 & L469, and/or H99 & R462.

In a specific embodiment, the stabilized HIV gp120 polypeptide or soluble gp140 polypeptide comprises an inner domain with an amino acid sequence that is at least a % identical to the inner domain sequence as set forth in SEQ ID NO: 2, wherein a is a value selected from 60%, 70%, 75%, 80%, 85%, 90%, 95%, 96%, 97%, 98%, 99% or greater, and comprises one or more pairs of non-naturally occurring cysteine residues at positions equivalent to position V59 & S109, V95 & W465, V95 & R462, V95 & L469, and/or H99 & R462 in SEQ ID NO: 2.

In a further embodiment, the stabilized HIV gp120 polypeptide or soluble gp140 polypeptide comprises an amino acid sequence with at least b % identity to the sequence as set forth in SEQ ID NO: 2, wherein b is a value selected from 60%, 70%, 75%, 80%, 85%, 90%, 95%, 96%, 97%, 98%, 99%, 99.5% or 99.9% or greater, and comprises one or more pairs of non-naturally occurring cysteine residues at positions equivalent to position V59 & S109, V95 & W465, V95 & R462, V95 & L469, and/or H99 & R462 in SEQ ID NO: 2.

In a further embodiment, the stabilized HIV gp120 polypeptide comprises an amino acid sequence as set forth in SEQ ID NOs: 4, 6, 8, 10, 12, 14, 16, 18, 20 or 22.

The stabilized HIV gp120 polypeptides or soluble gp140 polypeptide of the invention may also comprise further inter-domain and inter-layer stabilizations known in the art. For example, the stabilized HIV gp120 polypeptides of the invention may comprise cavity filling mutations as described in reference 4 or disulphide bonds as described in references 6 and 7. In particular, the stabilized HIV gp120 polypeptides of the invention may be additionally stabilized by a disulphide bond between one or more non-naturally encoded cysteine pairs at positions corresponding to W90 & E268, 1103 & Q413 in SEQ ID NO: 2.

The invention also provides immunogenic fragments of the polypeptides of the invention, wherein the immunogenic fragment comprises and simultaneously exposes both CD4-bound and CD4-binding site epitopes of HIV gp120. The immunogenic fragments may be at least 300, 350, 400, 450, 475, 480, 485, 490, 495 or 497 amino acids in length. In a particular embodiment, the immunogenic fragment comprises cysteine residues at positions equivalent to one or more of the following pairs in the wild-type SF162 HIV gp120 polypeptide: V59 & S109, V95 & W465, V95 & R462, V95 & L469, and/or H99 & R462.

A polypeptide of the invention may, compared to SEQ ID NO: 2 or SEQ ID NO: 23, include one or more (e.g. 1, 2, 3, 4, 5, 6, 7, 8, 9, 10, etc.) conservative amino acid substitutions as well as cysteine residues at one or more of the following pairs: V59 & S109, V95 & W465, V95 & R462, V95 & L469, H99 & R462. A polypeptide of the invention may, compared to SEQ ID NO:4, 6, 8, 10, 12, 14, 16, 18, 20 or 22, include one or more (e.g. 1, 2, 3, 4, 5, 6, 7, 8, 9, 10, etc.) conservative amino acid substitutions. A conservative amino acid substitution is defined as the replacements of one amino acid with another which has a related side chain, provided that the amino acid substitution does not reduce the stabilization of the gp120 polypeptide, i.e. the polypeptide is still stabilized in a conformation which simultaneously exposes both CD4-bound and CD4-binding site epitopes. Genetically-encoded amino acids are generally divided into four families: (1) acidic i.e. aspartate, glutamate; (2) basic i.e. lysine, arginine, histidine; (3) non-polar i.e. alanine, valine, leucine, isoleucine, proline, phenylalanine, methionine, tryptophan; and (4) uncharged polar i.e. glycine, asparagine, glutamine, cysteine, serine, threonine, tyrosine. Phenylalanine, tryptophan, and tyrosine are sometimes classified jointly as aromatic amino acids. In general, substitution of single amino acids within these families does not have a major effect on the biological activity. Moreover, the polypeptides may have one or more (e.g. 1, 2, 3, 4, 5, 6, 7, 8, 9, 10, etc.) single amino acid deletions relative to SEQ ID NO: 2, 4, 6, 8, 10, 12, 14, 16, 18, 20, 22 or 23. Furthermore, the polypeptides may include one or more (e.g. 1, 2, 3, 4, 5, 6, 7, 8, 9, 10, etc.) insertions (e.g. each of 1, 2, 3, 4 or 5 amino acids) relative to SEQ ID NO: 2, 4, 6, 8, 10, 12, 14, 16, 18, 20, 22 or 23. In embodiments that are stabilized by disulphide bonds, the deletions, insertions and substitutions may be at any position that does not interfere with the disulphide bond.

Polypeptides of the invention can be prepared in many ways e.g. by chemical synthesis (in whole or in part), by digesting longer polypeptides using proteases, by translation from RNA, by purification from cell culture (e.g. from recombinant expression), from the organism itself (e.g. after bacterial culture, or direct from patients), etc. A preferred method for production of peptides<40 amino acids long involves in vitro chemical synthesis [29, 30]. Solid-phase peptide synthesis is particularly preferred, such as methods based on tBoc or Fmoc [31] chemistry. Enzymatic synthesis [32] may also be used in part or in full. As an alternative to chemical synthesis, biological synthesis may be used e.g. the polypeptides may be produced by translation. This may be carried out in vitro or in vivo. Biological methods are in general restricted to the production of polypeptides based on L-amino acids, but manipulation of translation machinery (e.g. of aminoacyl tRNA molecules) can be used to allow the introduction of D-amino acids (or of other non natural amino acids, such as iodotyrosine or methylphenylalanine, azidohomoalanine, etc.) [33]. Where D-amino acids are included, however, it is preferred to use chemical synthesis. Polypeptides of the invention may have covalent modifications at the C-terminus and/or N-terminus.

Polypeptides of the invention are preferably provided in purified or substantially purified form i.e. substantially free from other polypeptides (e.g. free from naturally-occurring polypeptides), particularly from other HIV or host cell polypeptides, and are generally at least about 50% pure (by weight), and usually at least about 90% pure i.e. less than about 50%, and more preferably less than about 10% (e.g. 5% or less) of a composition is made up of other expressed polypeptides.

Polypeptides of the invention may be attached to a solid support. Polypeptides of the invention may comprise a detectable label (e.g. a radioactive or fluorescent label, or a biotin label).

The term “polypeptide” refers to amino acid polymers of any length and includes glycoproteins among other modifications and variants. HIV gp120 and or soluble gp140 are glycoproteins and the polypeptides of the invention will preferably be glycosylated. The amino acid polymer may be linear or branched, it may comprise modified amino acids, and it may be interrupted by non-amino acids. The terms also encompass an amino acid polymer that has been modified naturally or by intervention; for example, disulfide bond formation, additional glycosylation, partial or complete decylcosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. Polypeptides can occur as single chains or associated chains. Polypeptides of the invention can be naturally or non-naturally glycosylated (i.e. the polypeptide has a glycosylation pattern that differs from the glycosylation pattern found in the corresponding naturally occurring polypeptide). The polypeptides of the invention may be isolated or purified.

The invention provides polypeptides comprising a sequence -X-Y- or -Y-X-, wherein: -X- is an amino acid sequence as defined above, i.e. a polypeptide of the invention, and -Y- is not a sequence as defined above i.e. the invention provides fusion proteins. In one particular embodiment -Y- is an influenza hemaglutanin polypeptide or any suitable protein or biological molecule which aids in the function of the polypeptides of the invention.

The invention also provides the polypeptides of the invention in multimeric form. In particular, the invention provides trimers made up of three subunits, wherein each subunit is a polypeptide, fusion protein or immunogenic fragment according to the invention. In one embodiment, the trimer comprises at least two identical subunits. In one particular embodiment, all three subunits are identical. In an alternative embodiment, all three subunits are different.

The invention provides a process for producing polypeptides of the invention, comprising the step of culturing a host cell of the invention under conditions which induce polypeptide expression.

The invention provides a process for producing a polypeptide of the invention, wherein the polypeptide is synthesised in part or in whole using chemical means.

### Nucleic Acids

The invention also provides nucleic acids encoding the polypeptides of the invention. The invention provides nucleic acids comprising nucleotide sequences having sequence identity to nucleotide sequences encoding the polypeptides of the invention. Such nucleic acids include those using alternative codons to encode the same amino acid.

The invention also provides nucleic acid which can hybridize to these nucleic acids. Hybridization reactions can be performed under conditions of different “stringency”. Conditions that increase stringency of a hybridization reaction are widely known and published in the art. Examples of relevant conditions include (in order of increasing stringency): incubation temperatures of 25° C., 37° C., 50° C., 55° C. and 68° C.; buffer concentrations of 10×SSC, 6×SSC, 1×SSC, 0.1×SSC (where SSC is 0.15 M NaCl and 15 mM citrate buffer) and their equivalents using other buffer systems; formamide concentrations of 0%, 25%, 50%, and 75%; incubation times from 5 minutes to 24 hours; 1, 2, or more washing steps; wash incubation times of 1, 2, or 15 minutes; and wash solutions of 6×SSC, 1×SSC, 0.1×SSC, or de-ionized water. Hybridization techniques and their optimization are well known in the art [e.g. see ref 34].

The invention includes nucleic acid comprising sequences complementary to these sequences (e.g. for antisense or probing, or for use as primers).

Nucleic acid according to the invention can take various forms (e.g. single-stranded, double-stranded, vectors, primers, probes, labelled etc.). Nucleic acids of the invention may be circular or branched, but will generally be linear. Unless otherwise specified or required, any embodiment of the invention that utilizes a nucleic acid may utilize both the double-stranded form and each of two complementary single-stranded forms which make up the double-stranded form. Primers and probes are generally single-stranded, as are antisense nucleic acids.

Nucleic acids of the invention are preferably provided in purified or substantially purified form i.e. substantially free from other nucleic acids (e.g. free from naturally-occurring nucleic acids), particularly from other pneumococcal or host cell nucleic acids, generally being at least about 50% pure (by weight), and usually at least about 90% pure. Nucleic acids of the invention are preferably pneumococcal nucleic acids.

Nucleic acids of the invention may be prepared in many ways e.g. by chemical synthesis (e.g. phosphoramidite synthesis of DNA) in whole or in part, by digesting longer nucleic acids using nucleases (e.g. restriction enzymes), by joining shorter nucleic acids or nucleotides (e.g. using ligases or polymerases), from genomic or cDNA libraries, etc.

Nucleic acid of the invention may be attached to a solid support (e.g. a bead, plate, filter, film, slide, microarray support, resin, etc.). Nucleic acid of the invention may be labelled e.g. with a radioactive or fluorescent label, or a biotin label. This is particularly useful where the nucleic acid is to be used in detection techniques e.g. where the nucleic acid is a primer or as a probe.

The term “nucleic acid” includes in general means a polymeric form of nucleotides of any length, which contain deoxyribonucleotides, ribonucleotides, and/or their analogs. It includes DNA, RNA, DNA/RNA hybrids. It also includes DNA or RNA analogs, such as those containing modified backbones (e.g. peptide nucleic acids (PNAs) or phosphorothioates) or modified bases. Thus the invention includes mRNA, tRNA, rRNA, ribozymes, DNA, cDNA, recombinant nucleic acids, branched nucleic acids, plasmids, vectors, probes, primers, etc.. Where nucleic acid of the invention takes the form of RNA, it may or may not have a 5′ cap.

Nucleic acids of the invention may be part of a vector i.e. part of a nucleic acid construct designed for transduction/transfection of one or more cell types. Vectors may be, for example, “cloning vectors” which are designed for isolation, propagation and replication of inserted nucleotides, “expression vectors” which are designed for expression of a nucleotide sequence in a host cell, “viral vectors” which is designed to result in the production of a recombinant virus or virus-like particle, or “shuttle vectors”, which comprise the attributes of more than one type of vector. Preferred vectors are plasmids. A “host cell” includes an individual cell or cell culture which can be or has been a recipient of exogenous nucleic acid. Host cells include progeny of a single host cell, and the progeny may not necessarily be completely identical (in morphology or in total DNA complement) to the original parent cell due to natural, accidental, or deliberate mutation and/or change. Host cells include cells transfected or infected in vivo or in vitro with nucleic acid of the invention.

Where a nucleic acid is DNA, it will be appreciated that “U” in a RNA sequence will be replaced by “T” in the DNA. Similarly, where a nucleic acid is RNA, it will be appreciated that “T” in a DNA sequence will be replaced by “U” in the RNA.

The term “complement” or “complementary” when used in relation to nucleic acids refers to Watson-Crick base pairing. Thus the complement of C is G, the complement of G is C, the complement of A is T (or U), and the complement of T (or U) is A. It is also possible to use bases such as I (the purine inosine) e.g. to complement pyrimidines (C or T).

Nucleic acids of the invention can be used, for example: to produce polypeptides in vitro or in vivo; as hybridization probes for the detection of nucleic acid in biological samples; to generate additional copies of the nucleic acids; to generate ribozymes or antisense oligonucleotides; as single-stranded DNA primers or probes; or as triple-strand forming oligonucleotides.

The invention provides a process for producing nucleic acid of the invention, wherein the nucleic acid is synthesised in part or in whole using chemical means.

The invention provides vectors comprising nucleotide sequences of the invention (e.g. cloning or expression vectors) and host cells transformed with such vectors.

### Immunogenic Compositions

The invention also provides an immunogenic composition. Immunogenic compositions of the invention comprise a polypeptide of the invention, an immunogenic fragment thereof, a fusion protein of the invention, a trimeric polypeptide of the invention, a polynucleotide of the invention and/or a combination thereof, which may be referred to herein as antigens. Such immunogenic compositions may be useful as vaccines. These vaccines may either be prophylactic (i.e. to prevent infection) or therapeutic (i.e. to treat infection), but will typically be prophylactic.

Typically, immunogenic compositions comprise further components in order to make them pharmaceutically acceptable. They will usually include components in addition to the antigens e.g. they typically include one or more pharmaceutical carrier(s) and/or excipient(s). A thorough discussion of such components is available in reference 35.

Immunogenic compositions will generally be administered to a mammal in aqueous form. Prior to administration, however, the composition may have been in a non-aqueous form. For instance, although some vaccines are manufactured in aqueous form, then filled and distributed and administered also in aqueous form, other vaccines are lyophilised during manufacture and are reconstituted into an aqueous form at the time of use. Thus a composition of the invention may be dried, such as a lyophilised formulation.

The immunogenic composition may include preservatives such as thiomersal or 2-phenoxyethanol. It is preferred, however, that the vaccine should be substantially free from (i.e. less than 5 μg/ml) mercurial material e.g. thiomersal-free. Vaccines containing no mercury are more preferred. Preservative-free vaccines are particularly preferred.

To control tonicity, it is preferred to include a physiological salt, such as a sodium salt. Sodium chloride (NaCl) is preferred, which may be present at between 1 and 20 mg/ml e.g. about 10±2 mg/ml NaCl. Other salts that may be present include potassium chloride, potassium dihydrogen phosphate, disodium phosphate dehydrate, magnesium chloride, calcium chloride, etc.

Immunogenic compositions will generally have an osmolality of between 200 mOsm/kg and 400 mOsm/kg, preferably between 240-360 mOsm/kg, and will more preferably fall within the range of 290-310 mOsm/kg.

Immunogenic compositions may include one or more buffers. Typical buffers include: a phosphate buffer; a Tris buffer; a borate buffer; a succinate buffer; a histidine buffer (particularly with an aluminum hydroxide adjuvant); or a citrate buffer. Buffers will typically be included in the 5-20 mM range.

The pH of a composition will generally be between 5.0 and 8.1, and more typically between 6.0 and 8.0 e.g. 6.5 and 7.5, or between 7.0 and 7.8.

The composition is preferably sterile. The composition is preferably non-pyrogenic e.g. containing <1 EU (endotoxin unit, a standard measure) per dose, and preferably <0.1 EU per dose. The composition is preferably gluten free.

The composition may include material for a single immunisation, or may include material for multiple immunisations (i.e. a ‘multidose’ kit). The inclusion of a preservative is preferred in multidose arrangements. As an alternative (or in addition) to including a preservative in multidose immunogenic compositions, the immunogenic compositions may be contained in a container having an aseptic adaptor for removal of material.

Immunogenic compositions of the invention may also comprise one or more immunoregulatory agents. Preferably, one or more of the immunoregulatory agents include one or more adjuvants, for example two, three, four or more adjuvants. e.g. as disclosed in references 36 and 37 (for example, an adjuvant comprising one or more aluminium salts, or comprising a submicron oil-in-water emulsion).

The immunogenic compositions may be prepared as injectables, either as liquid solutions or suspensions. Solid forms suitable for solution in, or suspension in, liquid vehicles prior to injection can also be prepared (e.g. a lyophilised composition or a spray-freeze dried composition). The immunogenic composition may be prepared for oral administration e.g. as a tablet or capsule, as a spray, or as a syrup (optionally flavoured). The immunogenic composition may be prepared for nasal or ocular administration e.g. as drops. The immunogenic composition may be in kit form, designed such that a combined composition is reconstituted just prior to administration to a patient. Such kits may comprise one or more antigens in liquid form and one or more lyophilised antigens.

Where a composition is to be prepared extemporaneously prior to use (e.g. where a component is presented in lyophilised form) and is presented as a kit, the kit may comprise two vials, or it may comprise one ready-filled syringe and one vial, with the contents of the syringe being used to reactivate the contents of the vial prior to injection.

Immunogenic compositions used as vaccines comprise an immunologically effective amount of antigen(s), as well as any other components, as needed. By ‘immunologically effective amount’, it is meant that the administration of that amount to an individual, either in a single dose or as part of a series, is effective for treatment or prevention. This amount varies depending upon the health and physical condition of the individual to be treated, age, the taxonomic group of individual to be treated (e.g. non-human primate, primate, etc.), the capacity of the individual's immune system to synthesise antibodies, the degree of protection desired, the formulation of the vaccine, the treating doctor's assessment of the medical situation, and other relevant factors. It is expected that the amount will fall in a relatively broad range that can be determined through routine trials.

### Methods of Treatment

An isolated polypeptide of the invention, an immunogenic fragment thereof, a fusion protein of the invention, a trimeric polypeptide of the invention, a polynucleotide of the invention, an immunogenic compositions of the invention and/or a combination thereof can be used in methods to generate an immune response in a subject and in methods of treating and preventing infection by HIV and/or AIDS in a subject. In a preferred embodiment, the subject is human.

The methods for generating an immune response in a subject comprise administering an isolated polypeptide of the invention, an immunogenic fragment thereof, a fusion protein of the invention, a trimeric polypeptide of the invention, a polynucleotide of the invention, an immunogenic composition of the invention and/or a combination thereof to the subject

The immune responses raised by the methods and uses of the invention will generally include an antibody response, preferably a protective antibody response. Methods for assessing antibody responses, neutralizing capability and protection after HIV vaccination are well known in the art, for example as described in reference 38, 39, 40, 41 and 42.

The invention also provides a method of treating or preventing infection with HIV and/or AIDS comprising in a subject, comprising administering an isolated polypeptide of the invention, an immunogenic fragment thereof, a fusion protein of the invention, a trimeric polypeptide of the invention, a polynucleotide of the invention, an immunogenic compositions of the invention and/or a combination thereof to the subject.

Immunogenic compositions of the invention can be administered in various ways. The most preferred immunisation route is by intramuscular injection (e.g. into the arm or leg), but other available routes include subcutaneous injection, intranasal[43-45], intradermal[46, 47], oral[48], transcutaneous, transdermal[49], etc. Intradermal and intranasal routes are attractive. Intradermal administration may involve a microinjection device e.g. with a needle about 1.5 mm long.

Treatment can be by a single dose schedule or a multiple dose schedule. Multiple doses may be used in a primary immunisation schedule and/or in a booster immunisation schedule. In a multiple dose schedule the various doses may be given by the same or different routes e.g. a parenteral prime and mucosal boost, a mucosal prime and parenteral boost, etc. Administration of more than one dose (typically two doses) is particularly useful in immunologically naïve patients e.g. for people who have never received an HIV vaccine before. Multiple doses will typically be administered at least 1 week apart (e.g. about 2 weeks, about 3 weeks, about 4 weeks, about 6 weeks, about 8 weeks, about 12 weeks, about 16 weeks, etc.).

Immunogenic compositions of the invention may be administered to patients at substantially the same time as (e.g. during the same medical consultation or visit to a healthcare professional) an antiretroviral compound, and in particular an antiretroviral compound active against HIV.

### General

The term “comprising” encompasses “including” as well as “consisting” e.g. a composition “comprising” X may consist exclusively of X or may include something additional e.g. X+Y.

The word “substantially” does not exclude “completely” e.g. a composition which is “substantially free” from Y may be completely free from Y. Where necessary, the word “substantially” may be omitted from the definition of the invention.

The term “about” in relation to a numerical value x is optional and means, for example, x+10%.

Identity between polypeptide sequences is preferably determined by the Smith-Waterman homology search algorithm as implemented in the MPSRCH program (Oxford Molecular), using an affine gap search with parameters gap open penalty=12 and gap extension penalty=1.

### BRIEF DESCRIPTION OF THE DRAWINGS

FIG. 1 A: Structure of the inner domain of HIV gp120 from HXBc2 strain and its organization into layers 1, 2 and 3.
B: Sequence of HIV-1 gp120 from SF162 strain (SEQ ID NO: 2) showing associated layers and domains. Inner domain is in black text and outer domain is in grey text. Amino acids contributing to layer 1 are single underlined, amino acids contributing to layer 2 are double underlined and amino acids contributing to layer 3 are underlined with a broken line.

FIG. 2 Comparison of cysteine substituted and native gp120 structures

FIG. 3 Targets for layer 1 and 2 disulfide bridging based on gp120 structure from HXBc2 strain.

FIG. 4 Targets for layer 2 and 3 disulfide bridging based on gp120 structure from HXBc2 strain.

FIG. 5 Sites of cysteine substitution shown in alignment of Hxb2-gp120 (from which structural information is derived) and SF162 (reference sequence). Sites targeted for cysteine substitution and disulfide bridging are shown in boxes with the sequence number.

FIG. 6 Comparison of expression of wild type and cysteine substituted gp120 in mammalian cells. Wild type, single and double mutated plasmid encoding SF162 gp120 were transfected to 293 T-cells and after 48 hr of culture, cells were harvested and lysed. Equal amount of cell-lysate were run on SDS-PAGE under reducing or non-reducing conditions and HIV-gp120 in the lysate was detected by western blotting using anti-gp120 antibody (2G12).

FIG. 7 A: a typical Coomassie stained PAGE gel showing purified SF162 gp120 and disulphide stabilized SF162 L1-SS-L2 gp120 (SEQ ID NO: 4) to show >90% purity of the proteins.
B: a typical Coomassie stained PAGE gel showing purified SF162 gp140 and disulphide stabilized SF162 L1-SS-L2 gp140 (V59C and S109C) to show >90% purity of the proteins.

FIG. 8 Surface Plasmon Resonance (SPR) analysis to determine the binding affinity of gp120 & gp120 L1-SS-L2 to receptor (soluble CD4, sCD4), mAb b12 (that binds to CD4-binding site) & mAb 17b (an antibody that binds post CD4-binding in a CD4 induced-manner). The experimental data for the various concentrations of gp120 proteins injected over each ligand is shown, as well as the 1:1 fitted curves.

FIG. 9 A: Antibody binding studies 2 week-post 1^{st} immunization (2wp1), 2 weeks-post 2^{nd} immunization (2wp2), 2 weeks-post 3^{rd} immunization (2wp3), 4 weeks-post 3^{rd} immunization (4wp3) and 8 weeks-post 3^{rd} immunization (8wp3) time-points
B: Avidity index all four immunogens

FIG. 10 Neutralization of pseudoviruses (cross-subtype) in TZM-b1 assay. A—gp120; B—gp120 L1-SS-L2; C—gp140; D—gp140 L1-SS-L2.

FIG. 11 Epitope-specificity of the antibodies generated by A—gp120; B—gp120 L1-SS-L2; C—gp140; D—gp140 L1-SS-L2.

### MODES FOR CARRYING OUT THE INVENTION

### Strategy for Stabilizing Inter-Layer Contacts

### Identification of Sites for Cysteine Mutation and Disulfide Bridging.

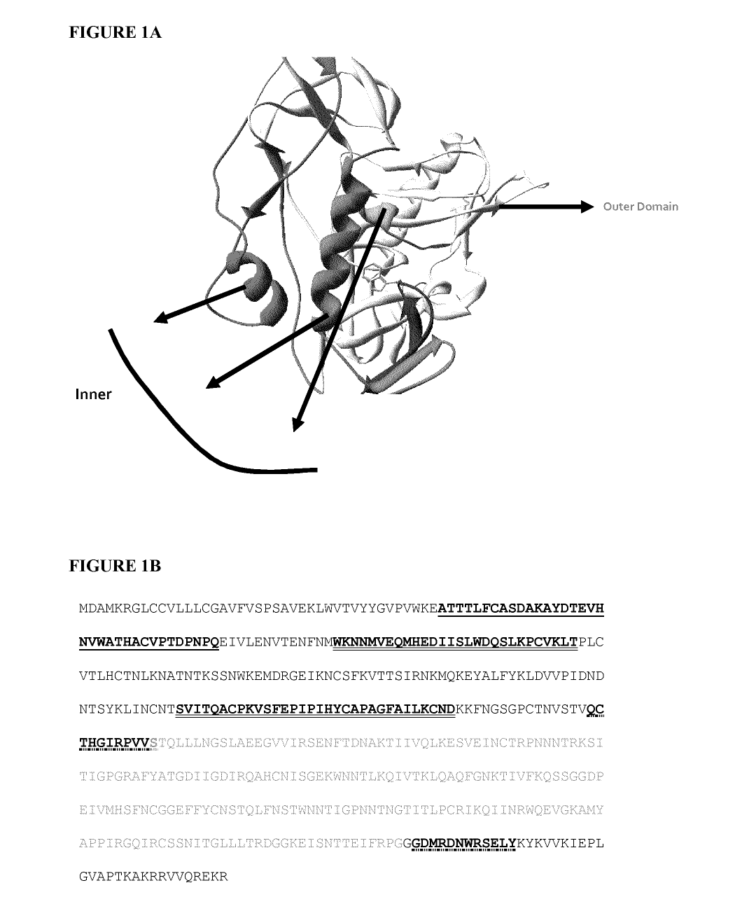

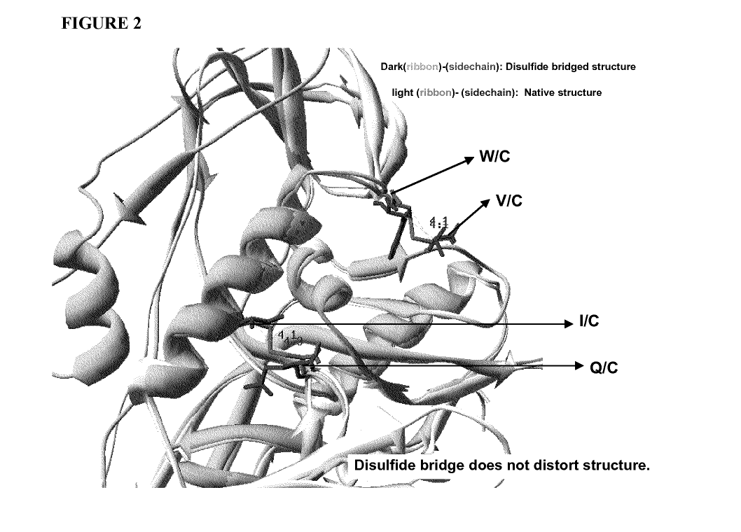

In order for a disulfide bridge to be formed between two cysteine residues, the side chains have to be at the proper distance and in the correct orientation with respect to each other. We have examined all residues that lie at the interface of layers 1, 2 and 3, and attempted to identify residues that can be targeted for substitution with cysteine for disulfide formation. We have used the crystal structure of gp120 [13] and the following criteria for this selection:
a. The average distances between Cβ (Carbon-beta) atoms of cysteines that formed disulfide bridges in previously resolved gp120 structures is about 4.22 Å, with the range 3.9 Å-4.7 Å. Therefore, we screened for residues on interacting layers that have Cβ-Cβ distances falling within this range.
b. We have also examined crystal structure of gp120 into which artificial disulfide bridges has been introduced by substitution with cysteine residues [6]. In this structure, two pairs of residues (W90-E268) and (I103-Q413) were substituted to cysteines (W90C-E268C) and (I103C-Q413C). The Cβ distances of the native and cysteine substituted residues were 4.1 Å and 4.3 Å, respectively. Furthermore, the disulfide bridges in the cysteine substituted residues were formed properly and the CD4-bound structure of these disulfide bridged gp120 was indistinguishable from the native gp120-CD4 bound structure (FIG. 2).

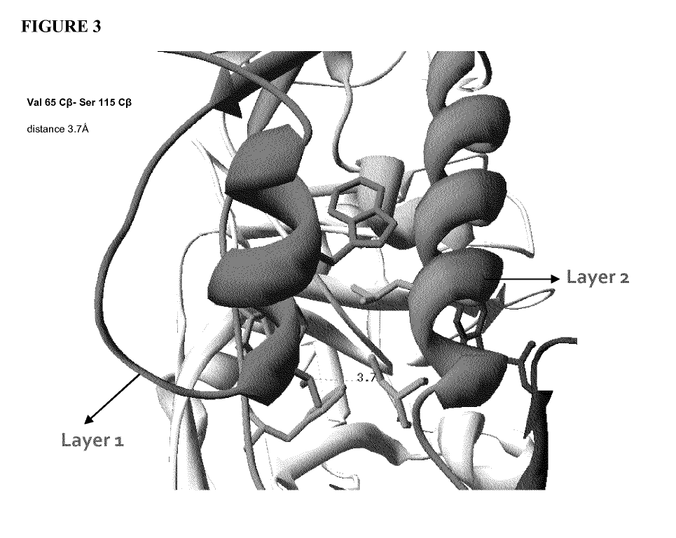

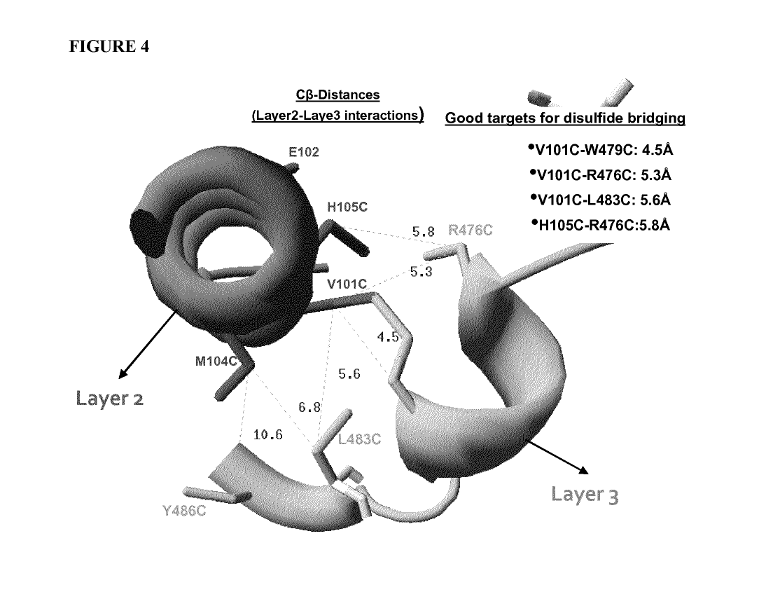

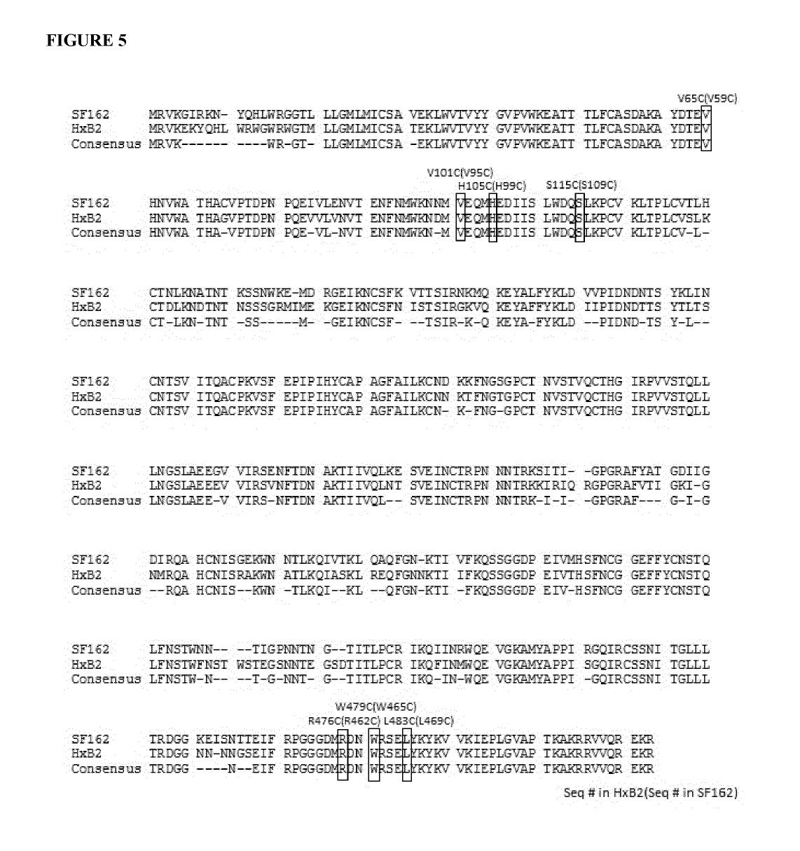

Based on these criteria we have identified multiple pairs of residues that if substituted to cysteines, will form disulfide bridges between layers (FIGS. 3, 4 & 5).

### Selection of Sites for Cysteine Substitution and Targeted Disulfide Bond Insertion

As described above, residues that lie at the interface of the layers and at close distance (approximately 4-5 angstroms), which if substituted with cysteine would be able to form disulfide bond were visually selected using the structural information available [13]. We have also used a software Disulfide-by-Design to further gain insight into residues that are presumed close enough if substituted by cysteine will be able to form a disulfide bond.

### Site Directed Mutagenesis for Cyteine Substitution

Once we decide on residues for cysteine substitution, we designed complementary primers according to Quick Change Site directed mutagenesis (Stratagen). The wild type SF162 gp120 coding plasmid is used as a template and after each round of mutagenesis clones were selected and sequenced to ensure presence of the correct mutation and absence of unintended mutations. The correct clones were further expanded for large scale plasmid DNA preparation.

### Targets for Layer 1 and Layer 2 Bridging

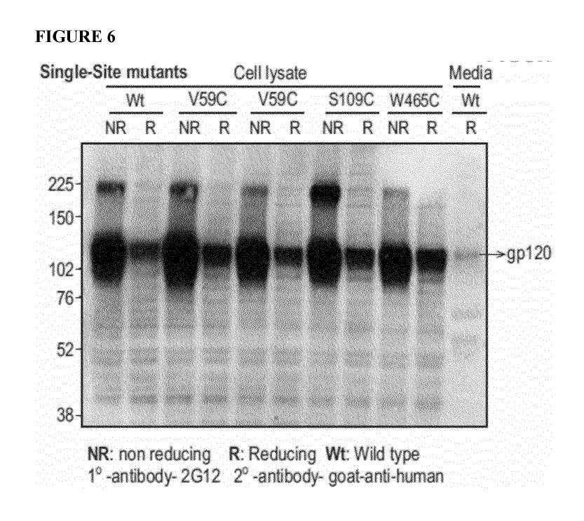

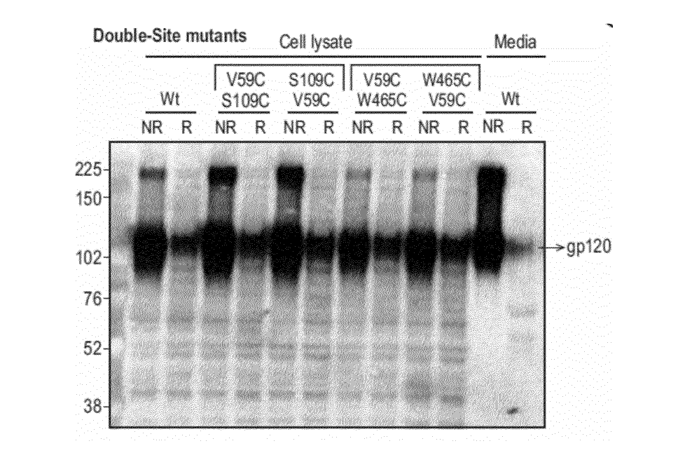

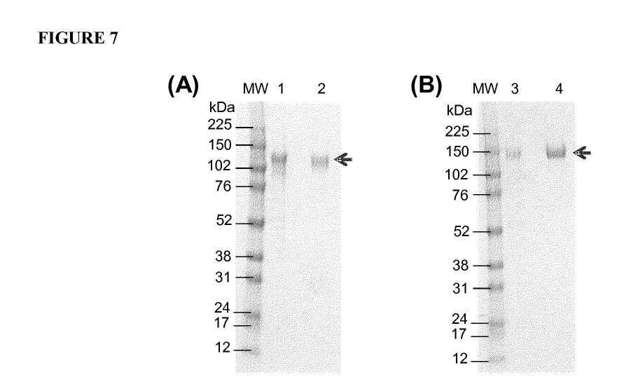

The targets chosen for cysteine substitution to bridge layer 1 and 2 are Valine 59 from layer 1 and Serine 109 from layer 2, (FIGS. 3 & 5). This bridge is referred to herein as L1-SS-L2. The Cβ atomic distances between these residues in the new CD4 bound gp120 structure is 3.7 Å, which is ideal for disulfide bridging. These two residues were mutated to cysteines by site directed mutagenesis using Stratagen® Quick Change protocol and confirmed by DNA sequencing (Table 1). Absence of deleterious effect in gp120 expression or processing associated with mutating these residues to cysteines was confirmed by small scale transfection and western blotting (FIG. 6).

TABLE 1

| | | | | | | | (control 2) |
|---|---|---|---|---|---|---|---|
| | | | | | | | W90C − V268C, |
| | Sequence | sequence | | | | | I103C − Q413C & |
| | numbering | numbering | | | | (control 1) | W90C − V268C − |
| | in HXBc2 | in SF162 | L1 − L2 | L2 − L3 | L1 − L2 − L3 | L1 & L3 | I103C − Q413C |
| 1 | V65C | V59C ✓ | V59C + S109C ✓ | | ✓ L1 − L2 + V95C + W465C | V59 + W465C | |
| | | | | | ✓ + R462C | | |
| | | | | | ✓ + L469C | | |
| 2 | V101C | V95C ✓ | | V95C + W465C ✓ | ✓ L2 − L3 + V59C + S109C | | |
| | | | | V95C + R462C ✓ | | | |
| | | | | V95C + L469C ✓ | | | |
| | | | | H99C + R462C | | | |
| 3 | S115C | S109C ✓ | S109C + V59C ✓ | | ✓ L1 − L2 + V95C + W465C | | |
| 4 | W479C | W465C ✓ | | W465C + V95C ✓ | | | |
| 5 | H105C | H99C ✓ | | | | | |
| 6 | R476C | R462C ✓ | | | | | |
| 7 | L483C | L469C ✓ | | | | | |
| 8 | W96C | W90C ✓ | | | | | ✓ W90C − V268C |
| | | | | | | | ✓ I103C − Q413C |
| | | | | | | | ✓ W90C − E268C − I130C |
| 9 | V275C | E268C ✓ | | | | | |
| 10 | I109C | I103C ✓ | | | | | |
| 11 | Q428C | Q413C ✓ | | | | | |

✓ Clones that are sequenced or being sequenced.

### Targets for Layer 2 and 3 Bridging

Based on similar criteria described above, we have identified a number of residues that lie on layers 2 and 3 that can be bridged by disulfide bond. These residues and the distances between the Cβ atoms listed below; V95-W465: 4.5 Å, V95-R462: 5.3 Å, V95-L469: 5.6 Å and H99-R462: 5.8 Å. Residues V95 and W465 will be the primary targets for cysteine mutation and disulfide bridging of layers 2 and 3, (FIGS. 4 & 5). The selected residues were mutated and the presence of mutation and absence of deleterious effect from the mutation was confirmed, as described above (Table 1 and FIG. 6).

### Combination Mutagenesis and Insertion of Multiple Disulfide Bonds

To achieve further stabilization we have proceeded with combination mutagenesis as indicated in Table 1 and described below. These include:
a. Combination of layers 1-2 and layers 2-3 double bridging using disulphide bonds between V59C & S109C and V95C & W465C
b. Combination of layer 1-2 bridge with the previously published, disulfide bridged gp120 structure [6] (W90C & E268C; 1103C & Q413C) using disulphide bonds between V59C & S109C.
c. Combination of layer 2-3 bridge with the previously published, disulfide bridged gp120 structure (W90C & E268C; 1103C & Q413C) [6] combined with disulphide bonds at V95C & W465C; W90C & E268C; or 1103C & Q413C.

### Protein Expression and Purification:

Recombinant HIV-1 envelope (Env) glycoproteins, gp120 and gp140, were derived from the subtype B CCR5-tropic strain HIV-1 SF162 and were produced by transfection of HEK293T cells. The loop 1 and 2 disulfide-stabilized gp120 (gp120 L1-SS-L2) and gp140 (gp140 L1-SS-L2) were also derived from SF162 and produced in HEK293T cells. All four glycoproteins were purified using a three-step purification process involving Galanthus Nivalis-Agarose (GNA) affinity column, cation-exchange DEAE column and a final ceramic hydroxyapatite (CHAP) column as described by Srivastava et al. [52]. Purified glycoproteins were then analyzed by SDS-PAGE (for level of purity) and immunoblots for specific reactivity (to anti-SF162 gp140 polyclonal rabbit sera). The purified glycoproteins were homogeneous (>95% monomer for gp120s; >80% trimer for gp140s) with purity of >98%. Endotoxin levels in glycoproteins were measured using Endosafe® cartridges and an Endosafe®-PTSTM spectrophotometer (Charles River Laboratories International, Inc., Wilmington, Mass.), and found to be ≦0.05 EU/immunization dose.

Other variants of SF162 gp120, used for epitope-mapping purposes, such as gp120ΔV3, gp120ΔV1V2, gp120 D368R (CD4-binding site mutant), gp120 1420R (CD4i site mutant) were also produced and purified, as described above and previously [50]. Purity of the protein is estimated from Coomassie stained SDS PAGE of the protein and Western blot.

FIG. 7A shows coomassie-stained SDS-PAGE of purified SF162 gp120 (lane 1) and disulfide-stabilized SF162 L1-SS-L2 gp120 (SEQ ID NO: 4) (lane 2) to show the >90% purity of the proteins. The arrow indicates the two 120 kDa proteins. FIG. 7B shows coomassie-stained SDS-PAGE of purified SF162 gp140 (lane 3) and disulfide-stabilized SF162 L1-SS-L2 gp140 (V59C and S109C) (lane 4) to show the >90% purity of the proteins (Panel B). The arrow indicates the two 140 kDa proteins.

MW refers to Molecular Weight marker; the molecular weights (in kDa) of each of bands in the marker are indicated.

### Characterization of the Protein by Surface Plasmon Resonance (SPR) Assay:

Structural change and conformational fixation of the purified protein from each of the mutants was assayed by SPR, which measured kinetics of binding to specific ligands. To determine the binding affinities of wild-type gp120 and the disulfide-stabilized gp120, gp120 L1-SS-L2, we used SPR-based BIAcore 3000. 200 RU of sCD4 or mAbs, b12 or 17b, were immobilized directly onto CM5 sensor chip via amine coupling. Varying concentrations of gp120s, either wild-type of disulfide-stabilized, were then injected at 80 μl/min. The binding analysis was performed at 25° C. with HBS-EP buffer as running buffer. The experimental curves were then fitted to a 1:1 Langmuir binding model BIAevaluation software 3.2 (BIAcore Inc). The association rate, Kon, dissociation rate, Koff, and the dissociation constant, Kd, which are derived following 1:1 fit are indicated in Table 2.

### Results

In order to characterize the effect of fixing the inter-layer mobility by disulfide bond on the structural conformation of gp120, protein from wild type (unchanged) and layers 1-2 disulfide linked mutants (V59C & S109C; L1-SS-L2) were purified and characterized by Surface Plamson Resonance (SPR) assay. In this assay, ligands that bind to different conformation of gp120 were captured on CM5 chips (see methods) and binding to wild type or mutant soluble gp120 was compared. Binding to 17b antibody, a CD4-induced antibody, which selectively binds to the CD4-bound conformation of gp120 was increased to the mutant gp120 by a significant amount compared to the wild type. Stabilization of interaction between layers 1 & 2 by disulfide bond led to more than 5 fold gain in affinity of binding to 17b. Interestingly, this gain in affinity is almost all derived from a gain in on-rate (see table 2), which confirms the fixation of gp120 in CD4-bound conformation. However, there still remains substantial amount of flexibility in gp120 stabilized by disulfide bond between layers 1 & 2 alone, which is particularly evident upon binding to soluble CD4.

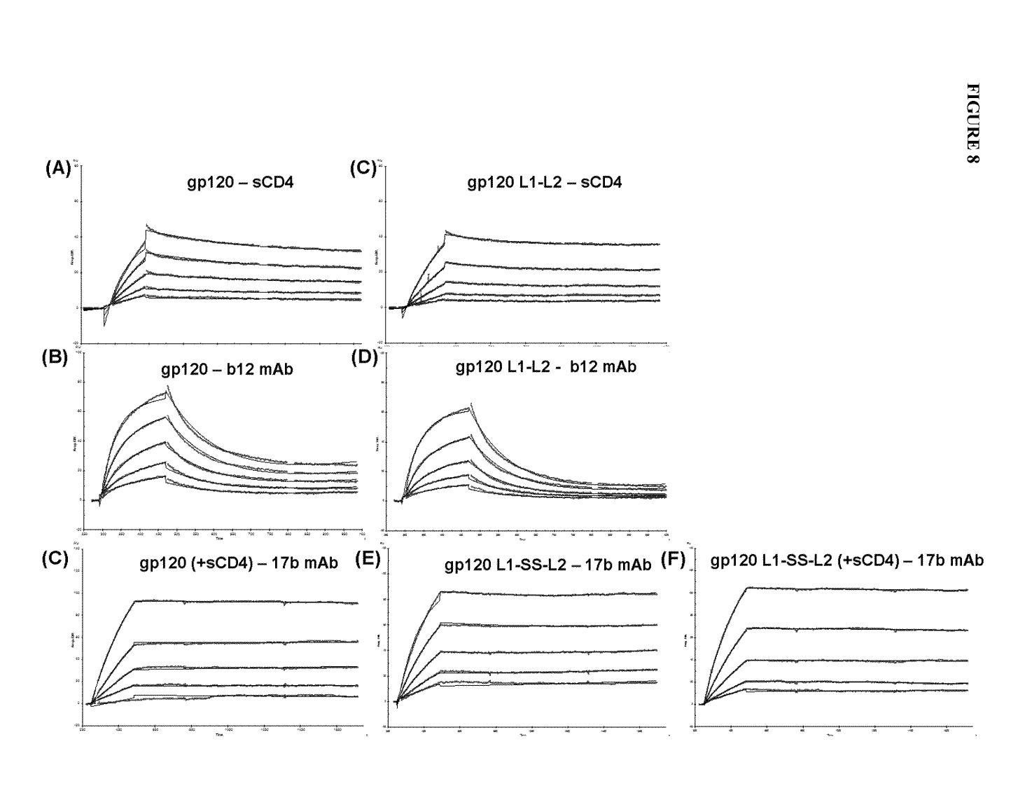

Surface Plasmon Resonance (SPR) analysis was also used to determine the binding affinity of gp120 & gp120 L1-SS-L2 to receptor (soluble CD4; sCD4), mAb b12 (that binds to CD4-binding site) as well as mAb 17b (FIG. 8). The lines denote the experimental data for the various concentrations of gp120 proteins injected over each ligand; the curved lines denote the 1:1 fitted curves. The near complete overlay of the two curves validates the goodness of fit (Chi2, Table 2) to 1:1 binding model.

TABLE 2

| Ligand- | Kₒₙ | K_{off} | K_{d} |
|---|---|---|---|
| analyte | (1/Ms) | (1/s) | (M) |
| Gp120 − sCD4 | 3.21e⁴ | 7.84e⁻⁴ | 2.45e⁻⁸ |
| Gp120 L1-SS-L2 − sCD4 | 8.02e³ | 7.46e⁻⁴ | 9.31e⁻⁸ |
| Gp120 − b12 | 1.65e⁵ | 7.12e⁻³ | 4.48e⁻⁸ |
| Gp120 L1-SS-L2 − b12 | 2.91e⁴ | 8.85e⁻³ | 3.04e⁻⁷ |
| Gp120 (+sCD4) − 17b | 2.04e⁵ | 5.35e⁻⁵ | 2.62e⁻¹⁰ |
| Gp120 L1-SS-L2 − 17b | 1.05e⁶ | 4.64e⁻⁴ | 4.42e⁻¹⁰ |
| Gp120 L1-SS-L2 | 3.46e⁵ | 3.33e⁻⁵ | 9.62e⁻¹¹ |
| (+sCD4) − 17b | | | |

Comparative kinetics analysis of binding of (wt/unmodified) gp120 and disulfide stabilized gp120 L1-SS-L2 show the following:
1. Binding to CD4: While gp120 bound soluble CD4 (sCD4) with K_{d} (dissociation constant) of 24.5 nM (2.48e⁻⁸M), the stabilized gp120 (gp120 L1-SS-L2) bound with 4-fold lower affinity (K_{d}=93.1 nM/9.31e⁻⁸M). This 4-fold difference was entirely due to lower on-rate (Kₒₙ) of the disulfide-stabilized gp120 to sCD4 because of “locking” the protein in CD4 bound conformation. [compare row 2 and 3].
2. Binding to b12: We observed a similar trend, but with greater fold difference≦7-fold, in K_{d} between gp120 and gp120 L1-SS-L2. [compare row 4 to 5]
3. Binding to 17b (+/−sCD4): We know gp120 does not bind efficiently to mAb 17b in absence of sCD4. However, in presence of sCD4 [row 6], gp120 bound with sub-nanomolar affinity of 0.26 nM (2.62 e⁻¹⁰M). Importantly and critically, the disulfide-stabilized gp120 (gp120 L1-SS-L2) bound mAb 17b in absence of sCD4 with affinity similar to that of gp120 in a CD4-bound state (gp120+CD4), 0.44 nM (4.42e⁻¹⁰M) [row 7]. Upon addition of sCD4 to the stabilized gp120, the affinity improves by 20-fold and shows a dissociation constant of 96 pM [9.62e⁻¹¹M, row 7].

In order to achieve complete stabilization of gp120 in the CD4-bound conformation further stabilizing mutations between layers 2 and 3 are being explored.

### Analysis of Candidate Immunogens

Once each further stabilized polypeptide is produced, it will be evaluated for the level of protein expression, proper folding and ligand binding. These will be compared to previously stabilized structures and other modified gp120s developed for enhanced exposure of the conserved binding sites. Those proteins that achieved stable exposure of conserved binding sites on gp120 will be further analyzed as candidate immunogens in small scale animal trials.

### Adjuvants-Carbopol971P NF+MF59 [51]

Carbopol® 971P NF (referred to as Carbopol 971P in this study) was purchased from Lubrizol as powder and was then resuspended in water under sterile conditions to generate a 0.5% homogenous, low viscosity suspension. The suspension was stored at 4° C. until further use. A 1:1 (v/v) mix of gp140 protein and 0.5% (w/v) Carbopol971P (pH≧3.0) was made for all in vitro evaluations. For administration in animals, a 1:1 (v/v) mix of gp140 and 0.5% (w/v) Carbopol971P was first made and the gp140 protein-Carbopol971P complex incubated for 30 minutes before addition of an equal volume of MF59, thereby keeping the final concentration of Carbopol971P suspension administered at 0.125% (w/v). For all Carbopol971P suspension for in vivo studies, endotoxin levels were measured using Endosafe® cartridges and an Endosafe®-PTSTM spectrophotometer (Charles River Laboratories International, Inc., Wilmington, Mass.).

### Rabbit Immunizations

Immunization studies were conducted at Josman LLC (Napa, Calif.), a research facility that is licensed through the USDA (No. 93-R-0260) and has a Public Health Service (PHS) Assurance from the NIH (No. A3404-01). Three groups of New Zealand White rabbits (5 young adult females per group) were used in the study. Rabbits were immunized with SF162 gp140 protein adjuvanted with either MF59 alone, Carbopol971P alone, or with Carbopol971P plus MF59. Four immunizations were administered intramuscularly, in the gluteus muscle (2 sites per immunization), at weeks 0, 4, 12, and 24. The protein dosage at each immunization was 50 μg. Serum samples were prepared from blood collected prior to the first immunization (pre-bleed) and at various time-points post each immunization (2wp2, 2wp3, 2wp4, 4wp4 and 15wp4) and analyzed for binding and neutralization.

For assessment of local reactogenicity associated with injection of 0.125% (w/v) Carbopol971P, visual observations of skin for edema and erythema at injection sites were performed pre-dose, immediately after immunization, and 24 h and 48 h after immunization. General observations for any obvious clinical signs were performed immediately after immunization, and 24 and 48 h post-immunization. Body-weights were also recorded before the beginning of the study, before each immunization, and 24 and 48 h following each immunization.

The study was fully approved by the Institutional Animal Care and Use Committee at Novartis (approval no. 09 NVD 044.3.3.09) in accordance with the requirements for the humane care and use of animals as set forth in the Animal Welfare Act, the ILAR Guide for the care and Use of Laboratory Animals, and all applicable local, state and federal laws and regulations.

### Results

We performed a rabbit study with protein-only (25 μg) immunization with Carbopol and MF59 as adjuvant. Wild-type gp120 and gp140 were compared to gp120 L1-SS-L2 (SEQ ID NO: 4) and gp140 L1-SS-L2 (V59C and S109C), respectively. Immunization was carried out as indicated in table 3 below.

TABLE 3

| Group | | Dose/ | | |
|---|---|---|---|---|
| (n = 5) | Immunogen | immunization | Weeks | Adjuvant |
| 1 | gP120 | 25 μg | 0, 4, 12 | Carbopol971P + |
| | | | | MF59 |
| 2 | gp120 L1- | 25 μg | 0, 4, 12 | Carbopol971P + |
| | SS-L2 | | | MF59 |
| 3 | gp140 | 25 μg | 0, 4, 12 | Carbopol971P + |
| | | | | MF59 |
| 4 | gp140 L1- | 25 μg | 0, 4, 12 | Carbopol971P + |
| | SS-L2 | | | MF59 |

Rabbits were inmmunised with the immunogen and adjuvant shown, at time points of 0, 4 weeks and 12 weeks.

Upon immunization, we evaluated binding Abs 2 week-post 1^{st} immunization (2wp1), 2w-post 2^{nd} immunization (2wp2), 2w-post 3^{rd} immunization (2wp3), 4wp3 and 8wp3 time-points. We observed that all four immunogens elicited >10e+5-6 GMT 2wp2/2wp3 immunization. Resuls are shown in FIG. 9A.

We also observed that all four immunogens generated similar levels of avidity at the five time-points analyzed except for gp140 L1-SS-L2, which at 2wp3 (2 week post final immunization) gave significantly higher avidity antibody. Results are shown in FIG. 9B.

### HIV-1 Virus Neutralization

We also analyzed the 2wp3 sera for neutralization of pseudoviruses (cross-subtype) in TZM-b1 assay. Virus neutralization titers were measured using a well-standardized assay employing pseudoviruses and a luciferase reporter gene assay in TZM-b1 cells [Dr. John C. Kappes, Dr. Xiaoyun Wu and Tranzyme, Inc. (Durham, N.C.)] as described previously [46, 47]. Briefly, a total of 200 TCID50 pseudoviruses/well were added to diluted serum samples and incubated at 37° C. for 1 h. Following incubation, 10,000 cells/well in DEAE-dextran-containing media were added and incubated for 48 h at 37° C. The final concentration of DEAE-dextran was 10 μg/ml. After a 48 h incubation, 100 μl of cells was transferred to a 96-well black solid plates (Costar) for measurements of luminescence using Bright Glo substrate solution as described by the supplier (Promega). Neutralization titers are the dilution at which relative luminescence units (RLU) were reduced by 50% compared to virus control wells after subtraction of background RLUs. HIV-1 Env pseudoviruses were prepared by co-transfection of 293T cells with expression plasmids containing full-length molecularly cloned gp160 env genes from a panel of HIV-1 isolates combined with an env-deficient HIV-1 backbone vector (pSG3Δenv) using FuGENE-6 HD (Roche Applied Sciences, Indianapolis, Ind.), as previously reported [Montefiori, D.C., Measuring HIV neutralization in a luciferase reporter gene assay. HIV Protocols: Second Edition ed. G. V. K. Vinayaka R. Prasad, eds. Vol. 485. 2009: Humana Press. 395-405]. After 48 h, the cell culture supernatants containing the pseudoviruses were filtered through a 0.45 μm filters and stored at −80° C. until use.

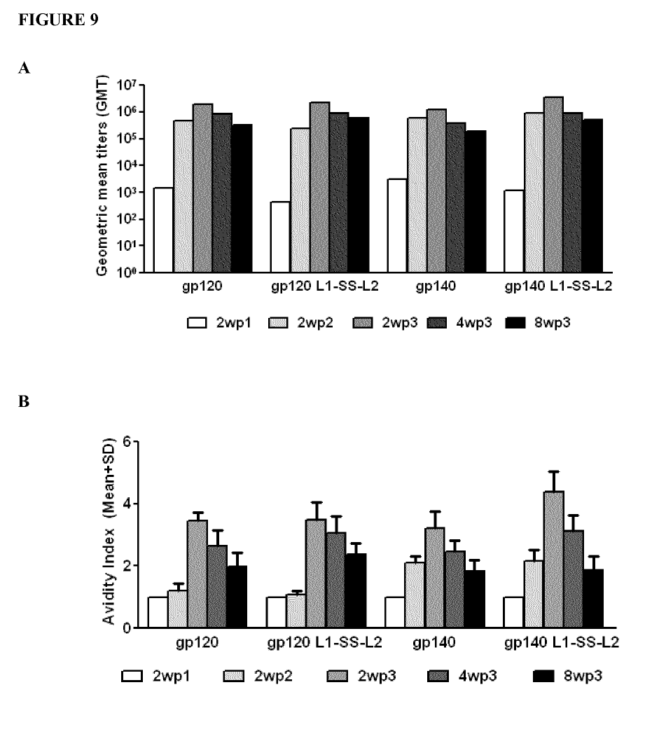

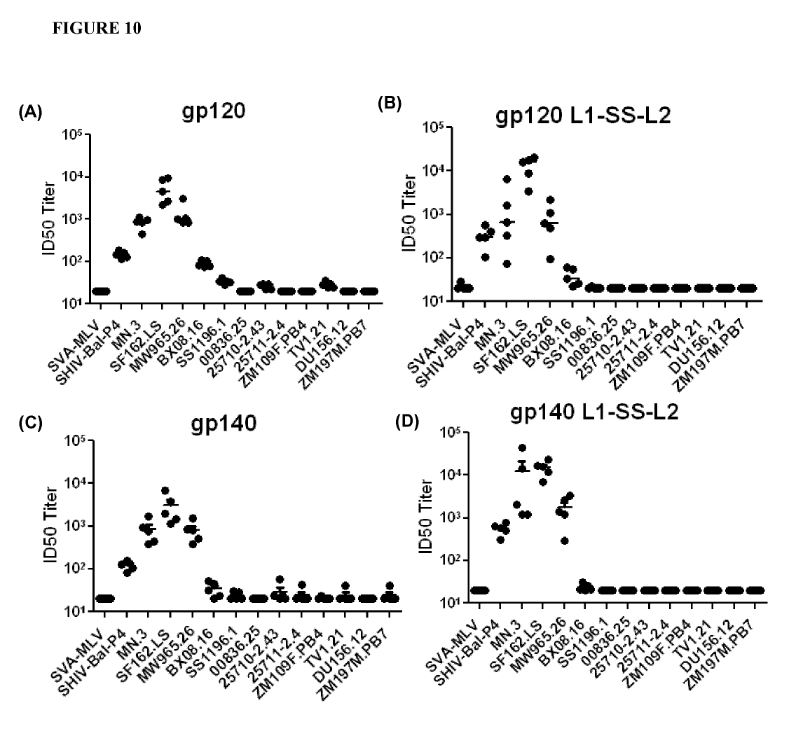

We observed that all four immunogens elicited Env-specific neutralizing antibodies against Tier 1a viruses [SHIV-Bal-P4(chimeric, subtype B), MN.3 (subtype B), SF162.LS (subtype B) and MW965.26 (subtype C)]. Importantly, both the disulfide-stabilized gp120 and gp140 generated >0.5 log higher homologous neutralizing titers (i.e., against SF162 virus). Results are shown in FIG. 10.

### Epitope Specificity

We used the 2wp3 sera to evaluate the epitope-specificity of the antibodies generated when immunized by the four immunogens. The four epitopes that we investigated were—V3 loop, V1V2 loop, CD4-binding site (CD4BS) & CD4 inducible/induced (CD4i; CD4 bound) site. Envelope-specific total antibody titers in sera from animals immunized with gp140 protein adjuvanted with MF59, gp140 protein adjuvanted with Carbopol971P, or gp140 protein adjuvanted with Carbopol971P plus MF59 were quantified by a standard ELISA assay using SF162 gp140 protein, as previously described [52]. Antibody avidity index determination was performed using an ammonium thiocyanate (NH₄SCN) displacement ELISA as described elsewhere [52].

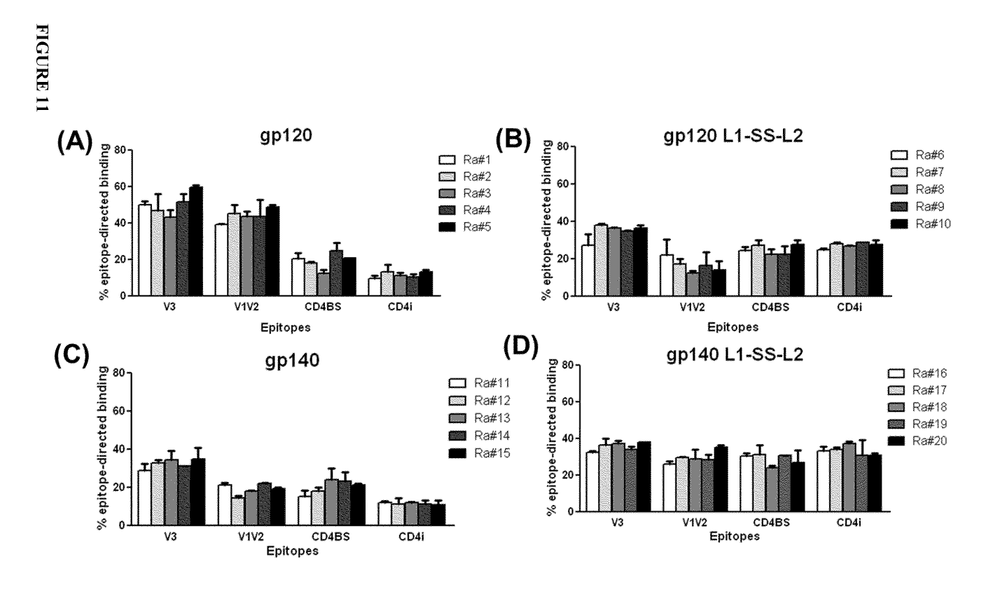

Upon analysis, we observed a significant difference in the quality of antibodies generated by the four immunogens. While gp120 elicited high levels of antibodies to V3 (˜50-55%) and V1V2-epitopes, antibodies directed to the more conserved CD4BS and CD4i-site were lower (10-20%) (FIG. 11A). However, the disulfide-stabilized gp120 generated significantly lower levels of antibodies to V3-(<40%) and V1V2-(15-20%) regions and comparatively higher levels of antibodies to CD4BS (≧30%; up from ˜20% in gp120-immunized arm) and CD4-site (>30%; up from 10% in gp120-immunized arm) (FIG. 11B).

Immunization using gp140 elicited <40% V3-reactive antibodies and ≦20%-V1V2 reactive antibodies. Antibodies to conserved CD4BS were ≧20% and to CD4i-site were ˜10% (FIG. 11C). However, when immunized with the disulfide-stabilized gp140, the 2wp3 exhibited similar levels of anti-V3 antibodies (<40%) but significantly higher levels of antibodies to V1V2 (30-35%), CD4BS (30-35%) and CD4i-site (≦40%) (FIG. 11D).

In conclusion, the disulfide-stabilized gp120 & gp140 generated significantly higher CD4i-site directed antibodies and the disulfide-stabilized gp140 elicited the most ‘balanced’ response to all epitopes of all.

Use of prime-boost regimen or more potent adjuvant may help increase the breadth or overall quality of the response.

It will be understood that the invention has been described by way of example only and modifications may be made whilst remaining within the scope and spirit of the invention.

### REFERENCES

[1] UNAIDS. 2008. UNAIDS Report on the global HIV/AIDS epidemic-2008. Report. UNAIDS.
[2] Johannes F. et al. (2009) Nature 458:636-640.
[3] Kwong D et al. (2002) Nature 420: 678-682.
[4] Kwong D et al. (2000) Structure 8: 1329-1339.
[5] Dey B et al (2009) PLoS Pathogens 5: el000445
[6] Zhou T et al. (2007) Nature. 445(7129):732-7.
[7] Chen L, et al. (2009) Science. 2009 326(5956):1123-7.
[8] Kwon et al. (2012) PNAS 109 (15) 5663-5668; published ahead of print Mar. 26, 2012, doi:10.1073/pnas.1112391109
[9] Kwong D et al. (1998) Nature. 393, 648-659.
[10] Chih-chin Huang, et al. (2005). Science 310: 1025-1028.
[11] Kassa A, et al. (2009). J Virol. 83:8364-78.
[12] Finzi A, et al (2010). Mol. Cell. 37:656-67.
[13] Pancera M, et al. (2010). Proc Natl Acad Sci 107:1166-71
[14] Chen B, et al (2005). Nature 433: 834-841.
[15] Kwong P (2005) Nature. 433(7028):834-41.
[16] Kassa A, et al. (2009). J. Virol. 83:4476-4488.
[17] Thali, M., et al., (1993) J Virol. 67:3978-3988
[18] Choe, H., et al (2003) Cell 114:161-170.
[19] Burton D R, et al (1994) Science 266(5187):1024-7.
[20] Posner M R, et al (1991) J Immunol. 146(12):4325-32
[21] Huang C C, et al.,(2007) Science 317(5846):1930-4
[22] Moore et al. (1994) J. Virol., 68:469-484
[23] Calarese D A, et al., (2003) Science. 300(5628):2065-71
[24] Karle S et al. (2004). AIDS 18(2):329-331.
[25] McKeating J et al. (1992) AIDS Res. Hum. Retroviruses. 8:451-459
[26] U.S. Pat. No. 6,241,986
[27] Xiang S et al. (2002) AIDS Res. Hum. Retrovir. 18:1207-1217
[28] Huang et al. (2004) PNAS; 101:2706-2711
[29] Bodanszky (1993) Principles of Peptide Synthesis (ISBN: 0387564314).
[30] Fields et al. (1997) Meth Enzymol 289: Solid-Phase Peptide Synthesis. ISBN: 0121821900.
[31] Chan & White (2000) Fmoc Solid Phase Peptide Synthesis. ISBN: 0199637245.
[32] Kullmann (1987) Enzymatic Peptide Synthesis. ISBN: 0849368413.
[33] Ibba (1996) Biotechnol Genet Eng Rev 13:197-216.
[34] U.S. Pat. No. 5,707,829
[35] Remington: The Science and Practice of Pharmacy (Gennaro, 2000; 20th edition, ISBN: 0683306472)
[36] Vaccine Design: The Subunit and Adjuvant Approach (eds. Powell & Newman) Plenum Press 1995 (ISBN 0-306-44867-X).
[37] Vaccine Adjuvants: Preparation Methods and Research Protocols (Volume 42 of Methods in Molecular Medicine series). ISBN: 1-59259-083-7. Ed. O'Hagan.
[38] Wei, X et al. (2003) Nature 422:307-312
[39] Montefiori, C (2004) Current Protocols in Immunology-John Wiley & Sons
[40] Barnett, et. al. (2010)J. Virol. p. 5975-5985, Vol. 84, No. 12.
[41] Burke B, et al. (2009) Virology 387(1):147-56
[42] Li et al (2005) J. Virol. 79:10108-10125
[43] Greenbaum et al. (2004) Vaccine 22:2566-77.
[44] Zurbriggen et al. (2003) Expert Rev Vaccines 2:295-304.
[45] Piascik (2003) J Am Pharm Assoc (Wash D.C.). 43:728-30.
[46] Halperin et al. (1979) Am J Public Health 69:1247-50.
[47] Herbert et al. (1979) J Infect Dis 140:234-8.
[48] Mann et al. (2004) Vaccine 22:2425-9.
[49] Chen et al. (2003) Vaccine 21:2830-6.
[50] AK Dey et al., PLoS One, 2012. 7(1): p. e30233
[51] AK Dey et al., Vaccine. 2012 Apr. 5; 30(17):2749-59. Epub 2012 Feb. 22)
[52] Srivastava, I. K., et al. J Virol, 2002. 76(6): p. 2835-47

1. An isolated polypeptide comprising (i) an HIV gp120 polypeptide, (ii) a soluble gp140 polypeptide, or (iii) an immunogenic fragment thereof, wherein:
said isolated polypeptide comprises a CD4-bound epitope and a CD4-binding site epitope; and
wherein both said CD4-bound and CD4-binding site epitopes are conformationally exposed on the surface of said isolated polypeptide.

2. The isolated polypeptide of claim 1, wherein the isolated polypeptide comprises an inner domain comprising layers 1, 2, and 3, and further comprises an inter-layer disulphide bond between said layers 1 and 2, or between said layers 2 and 3.

3. The isolated polypeptide of claim 2, wherein the isolated polypeptide comprises an inter-layer disulphide bond between layers 1 and 2.

4. The isolated polypeptide of claim 2, wherein the isolated polypeptide by comprises an inter-layer disulphide bond between layers 2 and 3.

5. The isolated polypeptide of claim 2, wherein the isolated polypeptide comprises an inter-layer disulphide bond between layers 1 and 2 and further comprises an inter-layer disulphide bond between layers 2 and 3.

6. The isolated polypeptide of claim 5, wherein the disulphide bond is formed between a pair of cysteine residues at positions equivalent to: V59C & S109C, V95C & W465C, V95C & R462C, V95C & L469C, H99C & R462C in SEQ ID NO: 2, or any combination thereof.

7. The isolated polypeptide of claim 1, wherein the isolated polypeptide (i) comprises an amino acid sequence with at least 90% identity to the inner domain sequence as set forth in SEQ ID NO: 2, and (ii) comprises one or more pairs of non-naturally occurring cysteine residues at positions equivalent to: V59 & S109, V95 & W465, V95 & R462, V95 & L469, H99 & R462 of SEQ ID NO: 2, or any combination thereof.

8. The isolated polypeptide of claim 1, wherein the isolated polypeptide (i) comprises an amino acid sequence with at least 90% identity to the sequence as set forth in SEQ ID NO: 2, and (ii) comprises one or more pairs of non-naturally occurring cysteine residues at positions equivalent to: V59 & S109, V95 & W465, V95 & R462, V95 & L469, H99 & R462 of SEQ ID NO: 2, or any combination thereof.

9. The isolated polypeptide of claim 1, wherein the isolated polypeptide comprises one or more non-naturally encoded cysteine pairs at positions corresponding to W90 & E268, 1103 & Q413 in SEQ ID NO: 2, or a combination thereof.

10. The isolated polypeptide of claim 1, wherein the isolated polypeptide comprises an amino acid sequence as set forth in SEQ ID NOs: 4, 6, 8, 10, 12, 14, 16, 18, 20 or 22.

11. (canceled)

12. A fusion protein comprising the isolated polypeptide claim 1 and a second polypeptide sequence.

13. The fusion protein of claim 12, wherein the second polypeptide sequence comprises a HA polypeptide sequence from influenza.

14. A trimer comprising three protein subunits, each subunit comprising the isolated polypeptide of claim 1.

15. A isolated polynucleotide encoding the isolated polypeptide of claim 1.

16. The polynucleotide of claim 15, wherein the polynucleotide comprises a nucleic acid sequence with at least 90% identity to any one of the sequences selected from the group consisting of: SEQ ID NOs: 3, 5, 7, 9, 11, 13, 15, 17, 19 and 21.

17. An immunogenic composition comprising the isolated polypeptide of claim 1.

18. The immunogenic composition of claim 17, further comprising an adjuvant.

19. (canceled)

20. A method for generating an immune response in a subject, comprising administering the isolated polypeptide of claim 1 to the subject.

21. (canceled)

22. A method of treating infection with HIV in a subject, comprising administering the isolated polypeptide of claim 1.

23. The method of claim 22, wherein the subject is human.


SEQUENCE LISTING (23 sequences)

SEQ ID NO: 1 - DNA, 1494 bp, HIV-1
atggatgcaatgaagagagggctctgctgtgtgctgctgctgtgtggagcagtcttcgtttcgcccagcgccgtggagaagctgtgggtgaccgtgtactacggcgtgcccgtgtggaaggaggccaccaccaccctgttctgcgccagcgacgccaaggcctacgacaccgaggtgcacaacgtgtgggccacccacgcctgcgtgcccaccgaccccaacccccaggagatcgtgctggagaacgtgaccgagaacttcaacatgtggaagaacaacatggtggagcagatgcacgaggacatcatcagcctgtgggaccagagcctgaagccctgcgtgaagctgacccccctgtgcgtgaccctgcactgcaccaacctgaagaacgccaccaacaccaagagcagcaactggaaggagatggaccgcggcgagatcaagaactgcagcttcaaggtgaccaccagcatccgcaacaagatgcagaaggagtacgccctgttctacaagctggacgtggtgcccatcgacaacgacaacaccagctacaagctgatcaactgcaacaccagcgtgatcacccaggcctgccccaaggtgagcttcgagcccatccccatccactactgcgcccccgccggcttcgccatcctgaagtgcaacgacaagaagttcaacggcagcggcccctgcaccaacgtgagcaccgtgcagtgcacccacggcatccgccccgtggtgagcacccagctgctgctgaacggcagcctggccgaggagggcgtggtgatccgcagcgagaacttcaccgacaacgccaagaccatcatcgtgcagctgaaggagagcgtggagatcaactgcacccgccccaacaacaacacccgcaagagcatcaccatcggccccggccgcgccttctacgccaccggcgacatcatcggcgacatccgccaggcccactgcaacatcagcggcgagaagtggaacaacaccctgaagcagatcgtgaccaagctgcaggcccagttcggcaacaagaccatcgtgttcaagcagagcagcggcggcgaccccgagatcgtgatgcacagcttcaactgcggcggcgagttcttctactgcaacagcacccagctgttcaacagcacctggaacaacaccatcggccccaacaacaccaacggcaccatcaccctgccctgccgcatcaagcagatcatcaaccgctggcaggaggtgggcaaggccatgtacgccccccccatccgcggccagatccgctgcagcagcaacatcaccggcctgctgctgacccgcgacggcggcaaggagatcagcaacaccaccgagatcttccgccccggcggcggcgacatgcgcgacaactggcgcagcgagctgtacaagtacaaggtggtgaagatcgagcccctgggcgtggcccccaccaaggccaagcgccgcgtggtgcagcgcgagaagcgctaa

SEQ ID NO: 2 - protein, 497 aa, HIV-1
  domain 1-248, 458-497: Inner Domain
MDAMKRGLCCVLLLCGAVFVSPSAVEKLWVTVYYGVPVWKEATTTLFCASDAKAYDTEVHNVWATHACVPTDPNPQEIVLENVTENFNMWKNNMVEQMHEDIISLWDQSLKPCVKLTPLCVTLHCTNLKNATNTKSSNWKEMDRGEIKNCSFKVTTSIRNKMQKEYALFYKLDVVPIDNDNTSYKLINCNTSVITQACPKVSFEPIPIHYCAPAGFAILKCNDKKFNGSGPCTNVSTVQCTHGIRPVVSTQLLLNGSLAEEGVVIRSENFTDNAKTIIVQLKESVEINCTRPNNNTRKSITIGPGRAFYATGDIIGDIRQAHCNISGEKWNNTLKQIVTKLQAQFGNKTIVFKQSSGGDPEIVMHSFNCGGEFFYCNSTQLFNSTWNNTIGPNNTNGTITLPCRIKQIINRWQEVGKAMYAPPIRGQIRCSSNITGLLLTRDGGKEISNTTEIFRPGGGDMRDNWRSELYKYKVVKIEPLGVAPTKAKRRVVQREKR

SEQ ID NO: 3 - DNA, 1494 bp, Artificial Sequence
  HIV-1 gp120 V59C and S109C
atggatgcaatgaagagagggctctgctgtgtgctgctgctgtgtggagcagtcttcgtttcgcccagcgccgtggagaagctgtgggtgaccgtgtactacggcgtgcccgtgtggaaggaggccaccaccaccctgttctgcgccagcgacgccaaggcctacgacaccgagtgccacaacgtgtgggccacccacgcctgcgtgcccaccgaccccaacccccaggagatcgtgctggagaacgtgaccgagaacttcaacatgtggaagaacaacatggtggagcagatgcacgaggacatcatcagcctgtgggaccagtgcctgaagccctgcgtgaagctgacccccctgtgcgtgaccctgcactgcaccaacctgaagaacgccaccaacaccaagagcagcaactggaaggagatggaccgcggcgagatcaagaactgcagcttcaaggtgaccaccagcatccgcaacaagatgcagaaggagtacgccctgttctacaagctggacgtggtgcccatcgacaacgacaacaccagctacaagctgatcaactgcaacaccagcgtgatcacccaggcctgccccaaggtgagcttcgagcccatccccatccactactgcgcccccgccggcttcgccatcctgaagtgcaacgacaagaagttcaacggcagcggcccctgcaccaacgtgagcaccgtgcagtgcacccacggcatccgccccgtggtgagcacccagctgctgctgaacggcagcctggccgaggagggcgtggtgatccgcagcgagaacttcaccgacaacgccaagaccatcatcgtgcagctgaaggagagcgtggagatcaactgcacccgccccaacaacaacacccgcaagagcatcaccatcggccccggccgcgccttctacgccaccggcgacatcatcggcgacatccgccaggcccactgcaacatcagcggcgagaagtggaacaacaccctgaagcagatcgtgaccaagctgcaggcccagttcggcaacaagaccatcgtgttcaagcagagcagcggcggcgaccccgagatcgtgatgcacagcttcaactgcggcggcgagttcttctactgcaacagcacccagctgttcaacagcacctggaacaacaccatcggccccaacaacaccaacggcaccatcaccctgccctgccgcatcaagcagatcatcaaccgctggcaggaggtgggcaaggccatgtacgccccccccatccgcggccagatccgctgcagcagcaacatcaccggcctgctgctgacccgcgacggcggcaaggagatcagcaacaccaccgagatcttccgccccggcggcggcgacatgcgcgacaactggcgcagcgagctgtacaagtacaaggtggtgaagatcgagcccctgggcgtggcccccaccaaggccaagcgccgcgtggtgcagcgcgagaagcgctaa

SEQ ID NO: 4 - protein, 497 aa, Artificial Sequence
  HIV-1 gp120 V59C and S109C
MDAMKRGLCCVLLLCGAVFVSPSAVEKLWVTVYYGVPVWKEATTTLFCASDAKAYDTECHNVWATHACVPTDPNPQEIVLENVTENFNMWKNNMVEQMHEDIISLWDQCLKPCVKLTPLCVTLHCTNLKNATNTKSSNWKEMDRGEIKNCSFKVTTSIRNKMQKEYALFYKLDVVPIDNDNTSYKLINCNTSVITQACPKVSFEPIPIHYCAPAGFAILKCNDKKFNGSGPCTNVSTVQCTHGIRPVVSTQLLLNGSLAEEGVVIRSENFTDNAKTIIVQLKESVEINCTRPNNNTRKSITIGPGRAFYATGDIIGDIRQAHCNISGEKWNNTLKQIVTKLQAQFGNKTIVFKQSSGGDPEIVMHSFNCGGEFFYCNSTQLFNSTWNNTIGPNNTNGTITLPCRIKQIINRWQEVGKAMYAPPIRGQIRCSSNITGLLLTRDGGKEISNTTEIFRPGGGDMRDNWRSELYKYKVVKIEPLGVAPTKAKRRVVQREKR

SEQ ID NO: 5 - DNA, 1494 bp, Artificial Sequence
  HIV-1 gp120 V95C and W465C
atggatgcaatgaagagagggctctgctgtgtgctgctgctgtgtggagcagtcttcgtttcgcccagcgccgtggagaagctgtgggtgaccgtgtactacggcgtgcccgtgtggaaggaggccaccaccaccctgttctgcgccagcgacgccaaggcctacgacaccgaggtgcacaacgtgtgggccacccacgcctgcgtgcccaccgaccccaacccccaggagatcgtgctggagaacgtgaccgagaacttcaacatgtggaagaacaacatgtgygagcagatgcacgaggacatcatcagcctgtgggaccagagcctgaagccctgcgtgaagctgacccccctgtgcgtgaccctgcactgcaccaacctgaagaacgccaccaacaccaagagcagcaactggaaggagatggaccgcggcgagatcaagaactgcagcttcaaggtgaccaccagcatccgcaacaagatgcagaaggagtacgccctgttctacaagctggacgtggtgcccatcgacaacgacaacaccagctacaagctgatcaactgcaacaccagcgtgatcacccaggcctgccccaaggtgagcttcgagcccatccccatccactactgcgcccccgccggcttcgccatcctgaagtgcaacgacaagaagttcaacggcagcggcccctgcaccaacgtgagcaccgtgcagtgcacccacggcatccgccccgtggtgagcacccagctgctgctgaacggcagcctggccgaggagggcgtggtgatccgcagcgagaacttcaccgacaacgccaagaccatcatcgtgcagctgaaggagagcgtggagatcaactgcacccgccccaacaacaacacccgcaagagcatcaccatcggccccggccgcgccttctacgccaccggcgacatcatcggcgacatccgccaggcccactgcaacatcagcggcgagaagtggaacaacaccctgaagcagatcgtgaccaagctgcaggcccagttcggcaacaagaccatcgtgttcaagcagagcagcggcggcgaccccgagatcgtgatgcacagcttcaactgcggcggcgagttcttctactgcaacagcacccagctgttcaacagcacctggaacaacaccatcggccccaacaacaccaacggcaccatcaccctgccctgccgcatcaagcagatcatcaaccgctggcaggaggtgggcaaggccatgtacgccccccccatccgcggccagatccgctgcagcagcaacatcaccggcctgctgctgacccgcgacggcggcaaggagatcagcaacaccaccgagatcttccgccccggcggcggcgacatgcgcgacaactgycgcagcgagctgtacaagtacaaggtggtgaagatcgagcccctgggcgtggcccccaccaaggccaagcgccgcgtggtgcagcgcgagaagcgctaa

SEQ ID NO: 6 - protein, 497 aa, Artificial Sequence
  HIV-1 gp120 V95C and W465C
MDAMKRGLCCVLLLCGAVFVSPSAVEKLWVTVYYGVPVWKEATTTLFCASDAKAYDTEVHNVWATHACVPTDPNPQEIVLENVTENFNMWKNNMCEQMHEDIISLWDQSLKPCVKLTPLCVTLHCTNLKNATNTKSSNWKEMDRGEIKNCSFKVTTSIRNKMQKEYALFYKLDVVPIDNDNTSYKLINCNTSVITQACPKVSFEPIPIHYCAPAGFAILKCNDKKFNGSGPCTNVSTVQCTHGIRPVVSTQLLLNGSLAEEGVVIRSENFTDNAKTIIVQLKESVEINCTRPNNNTRKSITIGPGRAFYATGDIIGDIRQAHCNISGEKWNNTLKQIVTKLQAQFGNKTIVFKQSSGGDPEIVMHSFNCGGEFFYCNSTQLFNSTWNNTIGPNNTNGTITLPCRIKQIINRWQEVGKAMYAPPIRGQIRCSSNITGLLLTRDGGKEISNTTEIFRPGGGDMRDNCRSELYKYKVVKIEPLGVAPTKAKRRVVQREKR

SEQ ID NO: 7 - DNA, 1494 bp, Artificial Sequence
  HIV-1 gp120 V95C and R462C
atggatgcaatgaagagagggctctgctgtgtgctgctgctgtgtggagcagtcttcgtttcgcccagcgccgtggagaagctgtgggtgaccgtgtactacggcgtgcccgtgtggaaggaggccaccaccaccctgttctgcgccagcgacgccaaggcctacgacaccgaggtgcacaacgtgtgggccacccacgcctgcgtgcccaccgaccccaacccccaggagatcgtgctggagaacgtgaccgagaacttcaacatgtggaagaacaacatgtgygagcagatgcacgaggacatcatcagcctgtgggaccagagcctgaagccctgcgtgaagctgacccccctgtgcgtgaccctgcactgcaccaacctgaagaacgccaccaacaccaagagcagcaactggaaggagatggaccgcggcgagatcaagaactgcagcttcaaggtgaccaccagcatccgcaacaagatgcagaaggagtacgccctgttctacaagctggacgtggtgcccatcgacaacgacaacaccagctacaagctgatcaactgcaacaccagcgtgatcacccaggcctgccccaaggtgagcttcgagcccatccccatccactactgcgcccccgccggcttcgccatcctgaagtgcaacgacaagaagttcaacggcagcggcccctgcaccaacgtgagcaccgtgcagtgcacccacggcatccgccccgtggtgagcacccagctgctgctgaacggcagcctggccgaggagggcgtggtgatccgcagcgagaacttcaccgacaacgccaagaccatcatcgtgcagctgaaggagagcgtggagatcaactgcacccgccccaacaacaacacccgcaagagcatcaccatcggccccggccgcgccttctacgccaccggcgacatcatcggcgacatccgccaggcccactgcaacatcagcggcgagaagtggaacaacaccctgaagcagatcgtgaccaagctgcaggcccagttcggcaacaagaccatcgtgttcaagcagagcagcggcggcgaccccgagatcgtgatgcacagcttcaactgcggcggcgagttcttctactgcaacagcacccagctgttcaacagcacctggaacaacaccatcggccccaacaacaccaacggcaccatcaccctgccctgccgcatcaagcagatcatcaaccgctggcaggaggtgggcaaggccatgtacgccccccccatccgcggccagatccgctgcagcagcaacatcaccggcctgctgctgacccgcgacggcggcaaggagatcagcaacaccaccgagatcttccgccccggcggcggcgacatgtcygacaactggcgcagcgagctgtacaagtacaaggtggtgaagatcgagcccctgggcgtggcccccaccaaggccaagcgccgcgtggtgcagcgcgagaagcgctaa

SEQ ID NO: 8 - protein, 497 aa, Artificial Sequence
  HIV-1 gp120 V95C and R462C
MDAMKRGLCCVLLLCGAVFVSPSAVEKLWVTVYYGVPVWKEATTTLFCASDAKAYDTEVHNVWATHACVPTDPNPQEIVLENVTENFNMWKNNMCEQMHEDIISLWDQSLKPCVKLTPLCVTLHCTNLKNATNTKSSNWKEMDRGEIKNCSFKVTTSIRNKMQKEYALFYKLDVVPIDNDNTSYKLINCNTSVITQACPKVSFEPIPIHYCAPAGFAILKCNDKKFNGSGPCTNVSTVQCTHGIRPVVSTQLLLNGSLAEEGVVIRSENFTDNAKTIIVQLKESVEINCTRPNNNTRKSITIGPGRAFYATGDIIGDIRQAHCNISGEKWNNTLKQIVTKLQAQFGNKTIVFKQSSGGDPEIVMHSFNCGGEFFYCNSTQLFNSTWNNTIGPNNTNGTITLPCRIKQIINRWQEVGKAMYAPPIRGQIRCSSNITGLLLTRDGGKEISNTTEIFRPGGGDMCDNWRSELYKYKVVKIEPLGVAPTKAKRRVVQREKR

SEQ ID NO: 9 - DNA, 1494 bp, Artificial Sequence
  HIV-1 gp120 V95C and L469C
atggatgcaatgaagagagggctctgctgtgtgctgctgctgtgtggagcagtcttcgtttcgcccagcgccgtggagaagctgtgggtgaccgtgtactacggcgtgcccgtgtggaaggaggccaccaccaccctgttctgcgccagcgacgccaaggcctacgacaccgaggtgcacaacgtgtgggccacccacgcctgcgtgcccaccgaccccaacccccaggagatcgtgctggagaacgtgaccgagaacttcaacatgtggaagaacaacatgtgygagcagatgcacgaggacatcatcagcctgtgggaccagagcctgaagccctgcgtgaagctgacccccctgtgcgtgaccctgcactgcaccaacctgaagaacgccaccaacaccaagagcagcaactggaaggagatggaccgcggcgagatcaagaactgcagcttcaaggtgaccaccagcatccgcaacaagatgcagaaggagtacgccctgttctacaagctggacgtggtgcccatcgacaacgacaacaccagctacaagctgatcaactgcaacaccagcgtgatcacccaggcctgccccaaggtgagcttcgagcccatccccatccactactgcgcccccgccggcttcgccatcctgaagtgcaacgacaagaagttcaacggcagcggcccctgcaccaacgtgagcaccgtgcagtgcacccacggcatccgccccgtggtgagcacccagctgctgctgaacggcagcctggccgaggagggcgtggtgatccgcagcgagaacttcaccgacaacgccaagaccatcatcgtgcagctgaaggagagcgtggagatcaactgcacccgccccaacaacaacacccgcaagagcatcaccatcggccccggccgcgccttctacgccaccggcgacatcatcggcgacatccgccaggcccactgcaacatcagcggcgagaagtggaacaacaccctgaagcagatcgtgaccaagctgcaggcccagttcggcaacaagaccatcgtgttcaagcagagcagcggcggcgaccccgagatcgtgatgcacagcttcaactgcggcggcgagttcttctactgcaacagcacccagctgttcaacagcacctggaacaacaccatcggccccaacaacaccaacggcaccatcaccctgccctgccgcatcaagcagatcatcaaccgctggcaggaggtgggcaaggccatgtacgccccccccatccgcggccagatccgctgcagcagcaacatcaccggcctgctgctgacccgcgacggcggcaaggagatcagcaacaccaccgagatcttccgccccggcggcggcgacatgcgcgacaactggcgcagcgagtgytacaagtacaaggtggtgaagatcgagcccctgggcgtggcccccaccaaggccaagcgccgcgtggtgcagcgcgagaagcgctaa

SEQ ID NO: 10 - protein, 497 aa, Artificial Sequence
  HIV-1 gp120 V95C and L469C
MDAMKRGLCCVLLLCGAVFVSPSAVEKLWVTVYYGVPVWKEATTTLFCASDAKAYDTEVHNVWATHACVPTDPNPQEIVLENVTENFNMWKNNMCEQMHEDIISLWDQSLKPCVKLTPLCVTLHCTNLKNATNTKSSNWKEMDRGEIKNCSFKVTTSIRNKMQKEYALFYKLDVVPIDNDNTSYKLINCNTSVITQACPKVSFEPIPIHYCAPAGFAILKCNDKKFNGSGPCTNVSTVQCTHGIRPVVSTQLLLNGSLAEEGVVIRSENFTDNAKTIIVQLKESVEINCTRPNNNTRKSITIGPGRAFYATGDIIGDIRQAHCNISGEKWNNTLKQIVTKLQAQFGNKTIVFKQSSGGDPEIVMHSFNCGGEFFYCNSTQLFNSTWNNTIGPNNTNGTITLPCRIKQIINRWQEVGKAMYAPPIRGQIRCSSNITGLLLTRDGGKEISNTTEIFRPGGGDMRDNWRSECYKYKVVKIEPLGVAPTKAKRRVVQREKR

SEQ ID NO: 11 - DNA, 1494 bp, Artificial Sequence
  HIV-1 gp120 H99C and R462C
atggatgcaatgaagagagggctctgctgtgtgctgctgctgtgtggagcagtcttcgtttcgcccagcgccgtggagaagctgtgggtgaccgtgtactacggcgtgcccgtgtggaaggaggccaccaccaccctgttctgcgccagcgacgccaaggcctacgacaccgaggtgcacaacgtgtgggccacccacgcctgcgtgcccaccgaccccaacccccaggagatcgtgctggagaacgtgaccgagaacttcaacatgtggaagaacaacatggtggagcagatgtgcgaggacatcatcagcctgtgggaccagagcctgaagccctgcgtgaagctgacccccctgtgcgtgaccctgcactgcaccaacctgaagaacgccaccaacaccaagagcagcaactggaaggagatggaccgcggcgagatcaagaactgcagcttcaaggtgaccaccagcatccgcaacaagatgcagaaggagtacgccctgttctacaagctggacgtggtgcccatcgacaacgacaacaccagctacaagctgatcaactgcaacaccagcgtgatcacccaggcctgccccaaggtgagcttcgagcccatccccatccactactgcgcccccgccggcttcgccatcctgaagtgcaacgacaagaagttcaacggcagcggcccctgcaccaacgtgagcaccgtgcagtgcacccacggcatccgccccgtggtgagcacccagctgctgctgaacggcagcctggccgaggagggcgtggtgatccgcagcgagaacttcaccgacaacgccaagaccatcatcgtgcagctgaaggagagcgtggagatcaactgcacccgccccaacaacaacacccgcaagagcatcaccatcggccccggccgcgccttctacgccaccggcgacatcatcggcgacatccgccaggcccactgcaacatcagcggcgagaagtggaacaacaccctgaagcagatcgtgaccaagctgcaggcccagttcggcaacaagaccatcgtgttcaagcagagcagcggcggcgaccccgagatcgtgatgcacagcttcaactgcggcggcgagttcttctactgcaacagcacccagctgttcaacagcacctggaacaacaccatcggccccaacaacaccaacggcaccatcaccctgccctgccgcatcaagcagatcatcaaccgctggcaggaggtgggcaaggccatgtacgccccccccatccgcggccagatccgctgcagcagcaacatcaccggcctgctgctgacccgcgacggcggcaaggagatcagcaacaccaccgagatcttccgccccggcggcggcgacatgtgygacaactggcgcagcgagctgtacaagtacaaggtggtgaagatcgagcccctgggcgtggcccccaccaaggccaagcgccgcgtggtgcagcgcgagaagcgctaa

SEQ ID NO: 12 - protein, 497 aa, Artificial Sequence
  HIV-1 gp120 H99C and R462C
MDAMKRGLCCVLLLCGAVFVSPSAVEKLWVTVYYGVPVWKEATTTLFCASDAKAYDTEVHNVWATHACVPTDPNPQEIVLENVTENFNMWKNNMVEQMCEDIISLWDQSLKPCVKLTPLCVTLHCTNLKNATNTKSSNWKEMDRGEIKNCSFKVTTSIRNKMQKEYALFYKLDVVPIDNDNTSYKLINCNTSVITQACPKVSFEPIPIHYCAPAGFAILKCNDKKFNGSGPCTNVSTVQCTHGIRPVVSTQLLLNGSLAEEGVVIRSENFTDNAKTIIVQLKESVEINCTRPNNNTRKSITIGPGRAFYATGDIIGDIRQAHCNISGEKWNNTLKQIVTKLQAQFGNKTIVFKQSSGGDPEIVMHSFNCGGEFFYCNSTQLFNSTWNNTIGPNNTNGTITLPCRIKQIINRWQEVGKAMYAPPIRGQIRCSSNITGLLLTRDGGKEISNTTEIFRPGGGDMCDNWRSELYKYKVVKIEPLGVAPTKAKRRVVQREKR

SEQ ID NO: 13 - DNA, 1494 bp, Artificial Sequence
  HIV-1 gp120 V59C S109C and V95C W465C
atggatgcaatgaagagagggctctgctgtgtgctgctgctgtgtggagcagtcttcgtttcgcccagcgccgtggagaagctgtgggtgaccgtgtactacggcgtgcccgtgtggaaggaggccaccaccaccctgttctgcgccagcgacgccaaggcctacgacaccgagtgccacaacgtgtgggccacccacgcctgcgtgcccaccgaccccaacccccaggagatcgtgctggagaacgtgaccgagaacttcaacatgtggaagaacaacatgtgygagcagatgcacgaggacatcatcagcctgtgggaccagtgcctgaagccctgcgtgaagctgacccccctgtgcgtgaccctgcactgcaccaacctgaagaacgccaccaacaccaagagcagcaactggaaggagatggaccgcggcgagatcaagaactgcagcttcaaggtgaccaccagcatccgcaacaagatgcagaaggagtacgccctgttctacaagctggacgtggtgcccatcgacaacgacaacaccagctacaagctgatcaactgcaacaccagcgtgatcacccaggcctgccccaaggtgagcttcgagcccatccccatccactactgcgcccccgccggcttcgccatcctgaagtgcaacgacaagaagttcaacggcagcggcccctgcaccaacgtgagcaccgtgcagtgcacccacggcatccgccccgtggtgagcacccagctgctgctgaacggcagcctggccgaggagggcgtggtgatccgcagcgagaacttcaccgacaacgccaagaccatcatcgtgcagctgaaggagagcgtggagatcaactgcacccgccccaacaacaacacccgcaagagcatcaccatcggccccggccgcgccttctacgccaccggcgacatcatcggcgacatccgccaggcccactgcaacatcagcggcgagaagtggaacaacaccctgaagcagatcgtgaccaagctgcaggcccagttcggcaacaagaccatcgtgttcaagcagagcagcggcggcgaccccgagatcgtgatgcacagcttcaactgcggcggcgagttcttctactgcaacagcacccagctgttcaacagcacctggaacaacaccatcggccccaacaacaccaacggcaccatcaccctgccctgccgcatcaagcagatcatcaaccgctggcaggaggtgggcaaggccatgtacgccccccccatccgcggccagatccgctgcagcagcaacatcaccggcctgctgctgacccgcgacggcggcaaggagatcagcaacaccaccgagatcttccgccccggcggcggcgacatgcgcgacaactgycgcagcgagctgtacaagtacaaggtggtgaagatcgagcccctgggcgtggcccccaccaaggccaagcgccgcgtggtgcagcgcgagaagcgctaa

SEQ ID NO: 14 - protein, 497 aa, Artificial Sequence
  HIV-1 gp120 V59C S109C and V95C W465C
MDAMKRGLCCVLLLCGAVFVSPSAVEKLWVTVYYGVPVWKEATTTLFCASDAKAYDTECHNVWATHACVPTDPNPQEIVLENVTENFNMWKNNMCEQMHEDIISLWDQCLKPCVKLTPLCVTLHCTNLKNATNTKSSNWKEMDRGEIKNCSFKVTTSIRNKMQKEYALFYKLDVVPIDNDNTSYKLINCNTSVITQACPKVSFEPIPIHYCAPAGFAILKCNDKKFNGSGPCTNVSTVQCTHGIRPVVSTQLLLNGSLAEEGVVIRSENFTDNAKTIIVQLKESVEINCTRPNNNTRKSITIGPGRAFYATGDIIGDIRQAHCNISGEKWNNTLKQIVTKLQAQFGNKTIVFKQSSGGDPEIVMHSFNCGGEFFYCNSTQLFNSTWNNTIGPNNTNGTITLPCRIKQIINRWQEVGKAMYAPPIRGQIRCSSNITGLLLTRDGGKEISNTTEIFRPGGGDMRDNCRSELYKYKVVKIEPLGVAPTKAKRRVVQREKR

SEQ ID NO: 15 - DNA, 1494 bp, Artificial Sequence
  HIV-1 gp120 V59C S109C and W90C E268C
atggatgcaatgaagagagggctctgctgtgtgctgctgctgtgtggagcagtcttcgtttcgcccagcgccgtggagaagctgtgggtgaccgtgtactacggcgtgcccgtgtggaaggaggccaccaccaccctgttctgcgccagcgacgccaaggcctacgacaccgagtgccacaacgtgtgggccacccacgcctgcgtgcccaccgaccccaacccccaggagatcgtgctggagaacgtgaccgagaacttcaacatgtgyaagaacaacatggtggagcagatgcacgaggacatcatcagcctgtgggaccagtgcctgaagccctgcgtgaagctgacccccctgtgcgtgaccctgcactgcaccaacctgaagaacgccaccaacaccaagagcagcaactggaaggagatggaccgcggcgagatcaagaactgcagcttcaaggtgaccaccagcatccgcaacaagatgcagaaggagtacgccctgttctacaagctggacgtggtgcccatcgacaacgacaacaccagctacaagctgatcaactgcaacaccagcgtgatcacccaggcctgccccaaggtgagcttcgagcccatccccatccactactgcgcccccgccggcttcgccatcctgaagtgcaacgacaagaagttcaacggcagcggcccctgcaccaacgtgagcaccgtgcagtgcacccacggcatccgccccgtggtgagcacccagctgctgctgaacggcagcctggccgaggagggcgtggtgatccgcagctgyaacttcaccgacaacgccaagaccatcatcgtgcagctgaaggagagcgtggagatcaactgcacccgccccaacaacaacacccgcaagagcatcaccatcggccccggccgcgccttctacgccaccggcgacatcatcggcgacatccgccaggcccactgcaacatcagcggcgagaagtggaacaacaccctgaagcagatcgtgaccaagctgcaggcccagttcggcaacaagaccatcgtgttcaagcagagcagcggcggcgaccccgagatcgtgatgcacagcttcaactgcggcggcgagttcttctactgcaacagcacccagctgttcaacagcacctggaacaacaccatcggccccaacaacaccaacggcaccatcaccctgccctgccgcatcaagcagatcatcaaccgctggcaggaggtgggcaaggccatgtacgccccccccatccgcggccagatccgctgcagcagcaacatcaccggcctgctgctgacccgcgacggcggcaaggagatcagcaacaccaccgagatcttccgccccggcggcggcgacatgcgcgacaactggcgcagcgagctgtacaagtacaaggtggtgaagatcgagcccctgggcgtggcccccaccaaggccaagcgccgcgtggtgcagcgcgagaagcgctaa

SEQ ID NO: 16 - protein, 497 aa, HIV-1 gp120 V59C S109C and W90C E268C
MDAMKRGLCCVLLLCGAVFVSPSAVEKLWVTVYYGVPVWKEATTTLFCASDAKAYDTECHNVWATHACVPTDPNPQEIVLENVTENFNMCKNNMVEQMHEDIISLWDQCLKPCVKLTPLCVTLHCTNLKNATNTKSSNWKEMDRGEIKNCSFKVTTSIRNKMQKEYALFYKLDVVPIDNDNTSYKLINCNTSVITQACPKVSFEPIPIHYCAPAGFAILKCNDKKFNGSGPCTNVSTVQCTHGIRPVVSTQLLLNGSLAEEGVVIRSCNFTDNAKTIIVQLKESVEINCTRPNNNTRKSITIGPGRAFYATGDIIGDIRQAHCNISGEKWNNTLKQIVTKLQAQFGNKTIVFKQSSGGDPEIVMHSFNCGGEFFYCNSTQLFNSTWNNTIGPNNTNGTITLPCRIKQIINRWQEVGKAMYAPPIRGQIRCSSNITGLLLTRDGGKEISNTTEIFRPGGGDMRDNWRSELYKYKVVKIEPLGVAPTKAKRRVVQREKR

SEQ ID NO: 17 - DNA, 1494 bp, Artificial Sequence
  HIV-1 gp120 V59C S109C and I103C Q413C
atggatgcaatgaagagagggctctgctgtgtgctgctgctgtgtggagcagtcttcgtttcgcccagcgccgtggagaagctgtgggtgaccgtgtactacggcgtgcccgtgtggaaggaggccaccaccaccctgttctgcgccagcgacgccaaggcctacgacaccgagtgccacaacgtgtgggccacccacgcctgcgtgcccaccgaccccaacccccaggagatcgtgctggagaacgtgaccgagaacttcaacatgtggaagaacaacatggtggagcagatgcacgaggacatctgyagcctgtgggaccagtgcctgaagccctgcgtgaagctgacccccctgtgcgtgaccctgcactgcaccaacctgaagaacgccaccaacaccaagagcagcaactggaaggagatggaccgcggcgagatcaagaactgcagcttcaaggtgaccaccagcatccgcaacaagatgcagaaggagtacgccctgttctacaagctggacgtggtgcccatcgacaacgacaacaccagctacaagctgatcaactgcaacaccagcgtgatcacccaggcctgccccaaggtgagcttcgagcccatccccatccactactgcgcccccgccggcttcgccatcctgaagtgcaacgacaagaagttcaacggcagcggcccctgcaccaacgtgagcaccgtgcagtgcacccacggcatccgccccgtggtgagcacccagctgctgctgaacggcagcctggccgaggagggcgtggtgatccgcagcgagaacttcaccgacaacgccaagaccatcatcgtgcagctgaaggagagcgtggagatcaactgcacccgccccaacaacaacacccgcaagagcatcaccatcggccccggccgcgccttctacgccaccggcgacatcatcggcgacatccgccaggcccactgcaacatcagcggcgagaagtggaacaacaccctgaagcagatcgtgaccaagctgcaggcccagttcggcaacaagaccatcgtgttcaagcagagcagcggcggcgaccccgagatcgtgatgcacagcttcaactgcggcggcgagttcttctactgcaacagcacccagctgttcaacagcacctggaacaacaccatcggccccaacaacaccaacggcaccatcaccctgccctgccgcatcaagcagatcatcaaccgctggtgygaggtgggcaaggccatgtacgccccccccatccgcggccagatccgctgcagcagcaacatcaccggcctgctgctgacccgcgacggcggcaaggagatcagcaacaccaccgagatcttccgccccggcggcggcgacatgcgcgacaactggcgcagcgagctgtacaagtacaaggtggtgaagatcgagcccctgggcgtggcccccaccaaggccaagcgccgcgtggtgcagcgcgagaagcgctaa

SEQ ID NO: 18 - protein, 497 aa, HIV-1 gp120 V59C S109C and I103C Q413C
MDAMKRGLCCVLLLCGAVFVSPSAVEKLWVTVYYGVPVWKEATTTLFCASDAKAYDTECHNVWATHACVPTDPNPQEIVLENVTENFNMWKNNMVEQMHEDICSLWDQCLKPCVKLTPLCVTLHCTNLKNATNTKSSNWKEMDRGEIKNCSFKVTTSIRNKMQKEYALFYKLDVVPIDNDNTSYKLINCNTSVITQACPKVSFEPIPIHYCAPAGFAILKCNDKKFNGSGPCTNVSTVQCTHGIRPVVSTQLLLNGSLAEEGVVIRSENFTDNAKTIIVQLKESVEINCTRPNNNTRKSITIGPGRAFYATGDIIGDIRQAHCNISGEKWNNTLKQIVTKLQAQFGNKTIVFKQSSGGDPEIVMHSFNCGGEFFYCNSTQLFNSTWNNTIGPNNTNGTITLPCRIKQIINRWCEVGKAMYAPPIRGQIRCSSNITGLLLTRDGGKEISNTTEIFRPGGGDMRDNWRSELYKYKVVKIEPLGVAPTKAKRRVVQREKR

SEQ ID NO: 19 - DNA, 1494 bp, Artificial Sequence
  HIV-1 gp120 V95C W465C and W90C E268C
atggatgcaatgaagagagggctctgctgtgtgctgctgctgtgtggagcagtcttcgtttcgcccagcgccgtggagaagctgtgggtgaccgtgtactacggcgtgcccgtgtggaaggaggccaccaccaccctgttctgcgccagcgacgccaaggcctacgacaccgaggtgcacaacgtgtgggccacccacgcctgcgtgcccaccgaccccaacccccaggagatcgtgctggagaacgtgaccgagaacttcaacatgtgyaagaacaacatgtgygagcagatgcacgaggacatcatcagcctgtgggaccagagcctgaagccctgcgtgaagctgacccccctgtgcgtgaccctgcactgcaccaacctgaagaacgccaccaacaccaagagcagcaactggaaggagatggaccgcggcgagatcaagaactgcagcttcaaggtgaccaccagcatccgcaacaagatgcagaaggagtacgccctgttctacaagctggacgtggtgcccatcgacaacgacaacaccagctacaagctgatcaactgcaacaccagcgtgatcacccaggcctgccccaaggtgagcttcgagcccatccccatccactactgcgcccccgccggcttcgccatcctgaagtgcaacgacaagaagttcaacggcagcggcccctgcaccaacgtgagcaccgtgcagtgcacccacggcatccgccccgtggtgagcacccagctgctgctgaacggcagcctggccgaggagggcgtggtgatccgcagctgyaacttcaccgacaacgccaagaccatcatcgtgcagctgaaggagagcgtggagatcaactgcacccgccccaacaacaacacccgcaagagcatcaccatcggccccggccgcgccttctacgccaccggcgacatcatcggcgacatccgccaggcccactgcaacatcagcggcgagaagtggaacaacaccctgaagcagatcgtgaccaagctgcaggcccagttcggcaacaagaccatcgtgttcaagcagagcagcggcggcgaccccgagatcgtgatgcacagcttcaactgcggcggcgagttcttctactgcaacagcacccagctgttcaacagcacctggaacaacaccatcggccccaacaacaccaacggcaccatcaccctgccctgccgcatcaagcagatcatcaaccgctggcaggaggtgggcaaggccatgtacgccccccccatccgcggccagatccgctgcagcagcaacatcaccggcctgctgctgacccgcgacggcggcaaggagatcagcaacaccaccgagatcttccgccccggcggcggcgacatgcgcgacaactgycgcagcgagctgtacaagtacaaggtggtgaagatcgagcccctgggcgtggcccccaccaaggccaagcgccgcgtggtgcagcgcgagaagcgctaa

SEQ ID NO: 20 - protein, 497 aa, Artificial Sequence
  HIV-1 gp120 V95C W465C and W90C E268C
MDAMKRGLCCVLLLCGAVFVSPSAVEKLWVTVYYGVPVWKEATTTLFCASDAKAYDTEVHNVWATHACVPTDPNPQEIVLENVTENFNMCKNNMCEQMHEDIISLWDQSLKPCVKLTPLCVTLHCTNLKNATNTKSSNWKEMDRGEIKNCSFKVTTSIRNKMQKEYALFYKLDVVPIDNDNTSYKLINCNTSVITQACPKVSFEPIPIHYCAPAGFAILKCNDKKFNGSGPCTNVSTVQCTHGIRPVVSTQLLLNGSLAEEGVVIRSCNFTDNAKTIIVQLKESVEINCTRPNNNTRKSITIGPGRAFYATGDIIGDIRQAHCNISGEKWNNTLKQIVTKLQAQFGNKTIVFKQSSGGDPEIVMHSFNCGGEFFYCNSTQLFNSTWNNTIGPNNTNGTITLPCRIKQIINRWQEVGKAMYAPPIRGQIRCSSNITGLLLTRDGGKEISNTTEIFRPGGGDMRDNCRSELYKYKVVKIEPLGVAPTKAKRRVVQREKR

SEQ ID NO: 21 - DNA, 1494 bp, Artificial Sequence
  HIV-1 gp120 V95C W465C and I103C Q413C
atggatgcaatgaagagagggctctgctgtgtgctgctgctgtgtggagcagtcttcgtttcgcccagcgccgtggagaagctgtgggtgaccgtgtactacggcgtgcccgtgtggaaggaggccaccaccaccctgttctgcgccagcgacgccaaggcctacgacaccgaggtgcacaacgtgtgggccacccacgcctgcgtgcccaccgaccccaacccccaggagatcgtgctggagaacgtgaccgagaacttcaacatgtggaagaacaacatgtgygagcagatgcacgaggacatctgyagcctgtgggaccagagcctgaagccctgcgtgaagctgacccccctgtgcgtgaccctgcactgcaccaacctgaagaacgccaccaacaccaagagcagcaactggaaggagatggaccgcggcgagatcaagaactgcagcttcaaggtgaccaccagcatccgcaacaagatgcagaaggagtacgccctgttctacaagctggacgtggtgcccatcgacaacgacaacaccagctacaagctgatcaactgcaacaccagcgtgatcacccaggcctgccccaaggtgagcttcgagcccatccccatccactactgcgcccccgccggcttcgccatcctgaagtgcaacgacaagaagttcaacggcagcggcccctgcaccaacgtgagcaccgtgcagtgcacccacggcatccgccccgtggtgagcacccagctgctgctgaacggcagcctggccgaggagggcgtggtgatccgcagcgagaacttcaccgacaacgccaagaccatcatcgtgcagctgaaggagagcgtggagatcaactgcacccgccccaacaacaacacccgcaagagcatcaccatcggccccggccgcgccttctacgccaccggcgacatcatcggcgacatccgccaggcccactgcaacatcagcggcgagaagtggaacaacaccctgaagcagatcgtgaccaagctgcaggcccagttcggcaacaagaccatcgtgttcaagcagagcagcggcggcgaccccgagatcgtgatgcacagcttcaactgcggcggcgagttcttctactgcaacagcacccagctgttcaacagcacctggaacaacaccatcggccccaacaacaccaacggcaccatcaccctgccctgccgcatcaagcagatcatcaaccgctggtgygaggtgggcaaggccatgtacgccccccccatccgcggccagatccgctgcagcagcaacatcaccggcctgctgctgacccgcgacggcggcaaggagatcagcaacaccaccgagatcttccgccccggcggcggcgacatgcgcgacaactgycgcagcgagctgtacaagtacaaggtggtgaagatcgagcccctgggcgtggcccccaccaaggccaagcgccgcgtggtgcagcgcgagaagcgctaa

SEQ ID NO: 22 - protein, 497 aa, Artificial Sequence
  HIV-1 gp120 V95C W465C and I103C Q413C
MDAMKRGLCCVLLLCGAVFVSPSAVEKLWVTVYYGVPVWKEATTTLFCASDAKAYDTEVHNVWATHACVPTDPNPQEIVLENVTENFNMWKNNMCEQMHEDICSLWDQSLKPCVKLTPLCVTLHCTNLKNATNTKSSNWKEMDRGEIKNCSFKVTTSIRNKMQKEYALFYKLDVVPIDNDNTSYKLINCNTSVITQACPKVSFEPIPIHYCAPAGFAILKCNDKKFNGSGPCTNVSTVQCTHGIRPVVSTQLLLNGSLAEEGVVIRSENFTDNAKTIIVQLKESVEINCTRPNNNTRKSITIGPGRAFYATGDIIGDIRQAHCNISGEKWNNTLKQIVTKLQAQFGNKTIVFKQSSGGDPEIVMHSFNCGGEFFYCNSTQLFNSTWNNTIGPNNTNGTITLPCRIKQIINRWCEVGKAMYAPPIRGQIRCSSNITGLLLTRDGGKEISNTTEIFRPGGGDMRDNCRSELYKYKVVKIEPLGVAPTKAKRRVVQREKR

SEQ ID NO: 23 - protein, 668 aa, HIV-1
MDAMKRGLCCVLLLCGAVFVSPSAVEKLWVTVYYGVPVWKEATTTLFCASDAKAYDTEVHNVWATHACVPTDPNPQEIVLENVTENFNMWKNNMVEQMHEDIISLWDQSLKPCVKLTPLCVTLHCTNLKNATNTKSSNWKEMDRGEIKNCSFKVTTSIRNKMQKEYALFYKLDVVPIDNDNTSYKLINCNTSVITQACPKVSFEPIPIHYCAPAGFAILKCNDKKFNGSGPCTNVSTVQCTHGIRPVVSTQLLLNGSLAEEGVVIRSENFTDNAKTIIVQLKESVEINCTRPNNNTRKSITIGPGRAFYATGDIIGDIRQAHCNISGEKWNNTLKQIVTKLQAQFGNKTIVFKQSSGGDPEIVMHSFNCGGEFFYCNSTQLFNSTWNNTIGPNNTNGTITLPCRIKQIINRWQEVGKAMYAPPIRGQIRCSSNITGLLLTRDGGKEISNTTEIFRPGGGDMRDNWRSELYKYKVVKIEPLGVAPTKAISSVVQSEKSAVTLGAMFLGFLGAAGSTMGARSLTLTVQARQLLSGIVQQQNNLLRAIEAQQHLLQLTVWGIKQLQARVLAVERYLKDQQLLGIWGCSGKLICTTAVPWNASWSNKSLDQIWNNMTWMEWEREIDNYTNLIYTLIEESQNQQEKNEQELLELDKWASLWNWFDISKWLWYI